(19) United States
(12) Patent Application Publication
(10) Pub. No.: US 2024/0108843 A1
(43) Pub. Date: Apr. 4, 2024

(54) SYSTEMS AND METHODS FOR IMPROVING PATIENT HEALTH

(71) Applicant: Belluscura LLC, Plano, TX (US)

(72) Inventor: Robert M. RAUKER, Plano, TX (US)

(21) Appl. No.: 18/536,937

(22) Filed: Dec. 12, 2023

Related U.S. Application Data
(63) Continuation-in-part of application No. 17/198,310, filed on Mar. 11, 2021.
Application No. 17/198,310 is a continuation-in-part of application No. PCT/US19/50496, filed on Sep. 11, 2019.
(60) Provisional application No. 62/729,664, filed on Sep. 11, 2018.

(51) Int. Cl. A61M 16/10; A61M 16/00; G16H 40/63

(52) U.S. Cl. CPC A61M 16/101; A61M 16/0003; A61M 16/026; G16H 40/63; A61M 2016/1025; A61M 2202/0007; A61M 2202/0208; A61M 2205/3303; A61M 2205/3327; A61M 2205/3553; A61M 2205/3561; A61M 2205/50; A61M 2205/8206; A61M 2230/005; A61M 2230/04; A61M 2230/10; A61M 2230/20; A61M 2230/42; A61M 2230/50; A61M 2230/63

(57) ABSTRACT

An integrated system for monitoring conditions of a patient and adjusting treatment for the patient based on comparisons of acquired data and historical data related to the patient includes a central processor, an oxygen concentrator, a level of consciousness monitor and a mobile device. The oxygen concentrator, the level of consciousness monitor and the mobile device are in communication with the central processor. The central processor includes a sleep database having baseline sleep information for a generic patient. The oxygen concentrator is configured to provide a flow of concentrated oxygen to the patient. The level of consciousness monitor is configured to collect data regarding the patient's state of wakefulness, awareness and alertness. The central processor collects data from the oxygen concentrator, the level of consciousness monitor and the mobile device and adjusts the oxygen concentrator based on comparisons of the baseline sleep information and the collected data.

### CROSS-REFERENCE TO RELATED APPLICATIONS

The present application is a continuation-in-part of U.S. patent application Ser. No. 17/198,310, filed Mar. 11, 2021 and titled, “Systems and Methods for Improving Patient Health,” which is a continuation-in-part under 35 U.S.C. §§ 120, 363 and 365(c) of International Patent Application No. PCT/US2019/050496, filed Sep. 11, 2019, which was published in English on Mar. 19, 2020 as International Publication No. WO 2020/055933, which claims the benefit of U.S. Provisional Patent Application No. 62/729,664, filed Sep. 11, 2018, and titled, “System and Method for Improving Patient Recovery Postoperatively,” the entire contents of which are incorporated herein by reference in their entirety.

### BACKGROUND OF THE INVENTION

Normal Sleep is important to maintain physical and mental health. Sleep disturbances frequently occur in patients after surgery. Factors associated with the development of postoperative sleep disturbances include old age, preoperative comorbidity, type of anesthesia, severity of surgical trauma, postoperative pain, environment stress, environment, postoperative medication, as well as other factors leading to discomfort of patients, including inability or restless sleep, which inhibit recovery. Development of sleep disturbances produces harmful effects on postoperative patients, that is, leading to higher risk of delirium, increased sensitivity to pain, more cardiovascular events, potential readmission and poor recovery. Both nonpharmacological and pharmacological measures (such as zolpidem, melatonin, and dexmedetomidine) can be used to improve postoperative sleep.

There are two main standards for the analysis of sleep, one published in 1968 by Rechtschaffen and Kales, the other in 2007 by the American Academy of Sleep Medicine. In the literature both standards are still used. Sleep can be divided into rapid eye movement (“REM”) sleep and non-rapid eye movement (“NREM”) sleep based on a person's electrophysiological patterns. REM sleep typically covers twenty to twenty-five percent (20-25%) of the total sleep time and is characterized by three main features: (1) a low voltage, fast frequency electroencephalogram (“EEG”) pattern that resembles an active, awake EEG pattern, (2) rapid eye movements, and (3) an atonic electromyogram (“EMG”) indicating inactivity of all voluntary muscles, except the extraocular muscles.

Atony or atonia is the result of direct inhibition of the alpha motor neurons. REM sleep is further divided into phasic REM sleep and tonic REM sleep. During phasic REM sleep there are bursts of rapid eye movements associated with brief burst of muscle activity, seen on EMG. Tonic REM sleep is the sleep between the phasic bursts where there is no or limited eye movement. Although REM sleep is typically a parasympathetic state, there is sympathetic activity during phasic REM sleep. The sudden increase in sympathetic activity gives rise to an increase in arterial blood pressure, heart rate and/or respiratory rate with an increased risk of cardiac ischemia, cerebral ischemia and cardiac arrhythmias. Short central apnoea's, hypopneas and long cardiac systoles have also been reported.

NREM sleep is also subdivided into different stages. The original standard of Rechtschaffen and Kales recognized four stages (N1 to N4), however, newer standards fuse stages N3 and N4 so that only three stages of NREM sleep are typically described. Stage N1 is the transition from wakefulness to sleep and is the lightest sleep stage. Stage N1 is characterized by low amplitude, relatively fast EEG frequencies in the theta range from four to seven hertz (4-7 Hz) and accounts for two to five percent (2-5%) of the total sleep time. Stage N2 sleep is called intermediate sleep and shows on EEG as a slowing of the frequency and an increase of the amplitude. This Stage N2 sleep accounts for forty to fifty percent (40-50%) of the total sleep time. Stage N3 is referred to as the deep sleep or slow wave sleep (“SWS”), which is characterized by low frequency, high amplitude delta EEG waves and accounts for twenty percent (20%) of the total sleep time. The sleep stages occur in ninety to one hundred twenty minute (90-120 min) cycles, with four to five cycles in a normal night. The first cycle starts with a brief passing from wakefulness to N1 sleep and then to stages N2 and N3. Subsequent cycles consist of N2, N3 and REM sleep. During the second half of the night, Stage N2 and REM sleep alternate with Stages N1 and N3 typically being absent.

Several structures within the brain are involved with sleep.

The hypothalamus, a peanut-sized structure deep inside the brain, contains groups of nerve cells that act as control centers affecting sleep and arousal. Within the hypothalamus is the suprachiasmatic nucleus (“SCN”)—clusters of thousands of cells that receive information about light exposure directly from the eyes and control behavioral rhythm. Some people with damage to the SCN sleep erratically throughout the day because they are not able to match their circadian rhythms with the light-dark cycle. Most blind people maintain some ability to sense light and are able to modify their sleep/wake cycle.

The brain stem, at the base of the brain, communicates with the hypothalamus to control the transitions between wake and sleep. (The brain stem includes structures called the pons, medulla, and midbrain). Sleep-promoting cells within the hypothalamus and the brain stem produce a brain chemical called gamma-Aminobutyric acid (“GABA”), which acts to reduce the activity of arousal centers in the hypothalamus and the brain stem. The brain stem (especially the pons and medulla) also plays a special role in REM sleep; as it sends signals to relax muscles essential for body posture and limb movements, so that we don't act out our dreams.

The thalamus acts as a relay for information from the senses to the cerebral cortex (the covering of the brain that interprets and processes information from short- to long-term memory). During most stages of sleep, the thalamus becomes quiet, letting you tune out the external world. But during REM sleep, the thalamus is active, sending the cortex images, sounds, and other sensations that fill our dreams.

The pineal gland, located within the brain's two hemispheres, receives signals from the SCN and increases production of the hormone melatonin, which helps put you to sleep once the lights go down. People who have lost their sight and cannot coordinate their natural wake-sleep cycle using natural light can stabilize their sleep patterns by taking small amounts of melatonin at the same time each day. Scientists believe that peaks and valleys of melatonin over time are important for matching the body's circadian rhythm to the external cycle of light and darkness.

The basal forebrain, near the front and bottom of the brain, also promotes sleep and wakefulness, while part of the midbrain acts as an arousal system. Release of adenosine (a chemical by-product of cellular energy consumption) from cells in the basal forebrain and probably other regions supports your sleep drive. Caffeine counteracts sleepiness by blocking the actions of adenosine.

The amygdala, an almond-shaped structure involved in processing emotions, becomes increasingly active during REM sleep.

There are two basic types of sleep: rapid eye movement (REM) sleep and non-REM sleep (which has three different stages). Each is linked to specific brain waves and neuronal activity. A person cycles through all stages of non-REM and REM sleep several times during a typical night, with increasingly longer, deeper REM periods occurring toward morning.

Stage 1 non-REM sleep is the changeover from wakefulness to sleep. During this short period (lasting several minutes) of relatively light sleep, a person's heartbeat, breathing, and eye movements slow, and muscles relax with occasional twitches. Brain waves begin to slow from their daytime wakefulness patterns.

Stage 2 non-REM sleep is a period of light sleep before entering deeper sleep. Heartbeat and breathing slow, and muscles relax even further. Body temperature drops and eye movements stop. Brain wave activity slows but is marked by brief bursts of electrical activity. People spend more of repeated sleep cycles in stage 2 sleep than in other sleep stages.

Stage 3 (and 4) non-REM sleep is the period of deep sleep that people need to feel refreshed in the morning. It occurs in longer periods during the first half of the night. Heartbeat and breathing slow to their lowest levels during this sleep stage. Muscles are relaxed and it may be difficult to awaken. Brain waves become even slower.

REM sleep first occurs about ninety minutes (90 min) after falling asleep. Your eyes move rapidly from side to side behind closed eyelids. Mixed frequency brain wave activity becomes closer to that seen in wakefulness. Breathing becomes faster and irregular, and your heart rate and blood pressure increase to near waking levels. Most dreaming occurs during REM sleep, although some can also occur in non-REM sleep. Arm and leg muscles become temporarily paralyzed, which prevents the sleeping individual from acting out dreams. As one ages, they spend less of their overall sleep time in REM sleep. Memory consolidation most likely requires both non-REM and REM sleep.

In the postoperative setting there are many different factors accountable for a disturbed sleep. For one, pain is a very important cause of disturbed sleep. Although assumed that pain relief is the most effective way to resolve this problem, thought must be given that pain medication on its own also disturbs the sleep architecture. The commonly used opioids have an irrefutable role in the postoperative changes in sleep architecture as proven by multiple independent studies. In addition, the question of what causes these changes in sleep patterns is more and more answered. Further, the postoperative sleep pattern is more severely disturbed than can be explained by opioids alone. In addition, even when opioids are completely avoided postoperatively, sleep disturbances remain. This observation favors the assumption that the biggest impact on sleep is seen as a result of surgical stress, tissue trauma and environmental factors. Due to the multitude of possible confounding factors during the postoperative setting, it remains difficult to separate the impact of each of these factors on postoperative sleep.

In sleep deprived patients, different physiologic changes have been reported. There are, for example, changes in respiratory, cardiovascular and endothelial disruptions caused by the release of inflammatory cytokines. After twenty-four to thirty hours (24-30 hrs.) of sleep deprivation, respiratory muscle weakness and a decreased ventilatory response to hypercapnia occurs. Sleep deprivation leads to an increased sympathetic and decreased parasympathetic tone and a state of increased catecholamine release, resulting in high blood pressure and heart rate and, as such, an increased risk of acute myocardial infarction. In animal settings, the necessity of sleep for an adequate immune response has been shown. Prolonged sleep deprivation onsets a catabolic state with opportunistic infections followed by septicaemia and death in less than a month. In humans, the relationship between sleep deprivation and immunology is less clear. Data suggest that sleep deprivation affects cellular immunity and cytokine function, but the exact mechanism and clinical implications are not known. There is a rise in cortisol levels and catecholamine release, reflected by the increased metabolic indices as oxygen consumption and carbon dioxide production. The same circumstances are present in patients with sepsis, which may suggest that sleep deprivation intensifies the stress response. Also, glucose metabolism is changed with a decreased sensitivity to insulin and impaired glucose tolerance.

There are also psychological changes caused by sleep deprivation. Delirium is the best know psychologic postoperative complication. Delirium can also be present in critically ill patients. Although the exact contribution of sleep deprivation to the development of delirium is not clear, both conditions share important mechanisms, risk factors and symptoms.

Sleep-related hypoxemia is a sleep-related breathing disorder characterized by abnormally low oxygen levels in the blood, usually related to sleep-disordered breathing patterns like hypoventilation, sleep apnea, or another type of breathing abnormality. Generally, oxygen saturation levels below ninety percent (90%) indicate hypoxemia, and levels below eighty percent (80%) are considered severe hypoxemia. Hypoxemia is sometimes called hypoxia.

In people with a sleep-related breathing disorder, nighttime breathing that's abnormally slow, shallow, or that stops and starts can result in too little oxygen in the blood. Sleep-related hypoxemia can also be triggered by environments that reduce the amount of oxygen available to the body, like high altitudes, air travel, or smoke.

Health conditions affecting the lungs, including chronic obstructive pulmonary disease (“COPD”), bronchitis, emphysema, lung cancer, pneumonia and asthma can increase the risk of hypoxemia. Certain narcotic pain relievers that alter breathing patterns can contribute to hypoxemia, including codeine, fentanyl, and morphine.

At higher altitudes above approximately three thousand seven hundred meters (3,700 m) or over twelve thousand fees (12,000 ft), increasing the concentration of oxygen a person breathes while they are sleeping has been shown to increase the amount of time a person spends in deep sleep or in Stages N3 and N4.

In addition to environmental, external and drug-related factors impacting sleep quality, diseases such as COPD, asthma, bronchitis, and other respiratory diseases can severely impact oxygen intake. Furthermore, there are other causes of sleep deprivation. In sleep, the upper airway muscle tone of the patients with sleep apnea tends to narrow and collapses temporarily. When this happens, the breathing stops accompanied by a drop in blood oxygen levels and arousal from sleep.

The low oxygen levels during sleep make sleep apnea patients very tired in the morning and will contribute to more restless sleep. Furthermore, when the oxygen levels start to drop, the carbon dioxide levels build up in their blood. This can lead to morning headaches, fatigue and sleepiness during the day. Any value of blood oxygen level below ninety-two percent (92%) is abnormal. However, the number of desaturations and the time spent with abnormal oxygen levels is important.

For example, if a person only desaturated below ninety-two percent (92%) once or twice during a seven hour (7 hr.) sleep, and the desaturation level lasted only a couple of seconds, it's typically not a reason for worry. If a doctor discovers that his patient's blood oxygen level (oxygen saturation) is less than about ninety percent (90%) during the day (when they are resting), then their oxygen levels are probably dropping during the night. This means that the patient likely has sleep apnea, or other respiratory disorders, like upper airway resistance syndrome, (UARS).

Although significant research has been performed on sleep, why we need it, what happens if we do not get enough of it, and the causes of sleep deprivation, there is still much that we do not understand. Notwithstanding this, there is a general consensus that sleep deprivation harms the body and can delay recovery or cause readmission of patients postoperatively.

A further problem with the evaluation of sleep is the difficulty of creating a laboratory environment where sleep studies can be performed with as little artificial interference as possible. For example, researchers agree that, for the most part, patients sleep better at home or in a less clinical environment, than in a hospital or clinic. Equipment for monitoring sleep, however, because of its cost, is typically located in the clinic or hospital. However, because a postoperative patient in recovery may be, for example, needing supplemental oxygen due to COPD, a continuous positive airway pressure (CPAP) machine to treat sleep apnea, medication or a combination of all three, currently, sleeping in a sleep clinic or in the presence of a healthcare provider may be necessary to coordinate the usage of these treatments to improve sleep. Providing an affordable system for remotely monitoring and controlling the various postoperative equipment and medication for postoperative patient or patients with breathing problems and/or other related medical issues is needed.

Artificial intelligence (“AI”) can be defined as the theory and development of computer systems able to perform tasks that normally require human intelligence, such as visual perception, speech recognition, decision-making, and translation between languages. AI is typically associated with a computer system's ability to “learn” and “problem solve,” based on experience with outcomes or collection and analysis of data related to a particular subject matter. AI has been used in medicine previously to prescribe treatments and predict treatment outcomes for various patients based on historical patient data.

It would be desirable to design, develop and deploy a system that addresses the shortcomings of monitoring and treating various postoperative sleep limitations of patients to improve and expedite recovery and to use such improvements to enhance the overall health of patients suffering from lung and heart disease where supplemental oxygen is prescribed. The preferred system of the present invention addresses the shortcomings of existing systems.

### BRIEF SUMMARY OF THE INVENTION

The preferred present invention relates to an integrated system for monitoring conditions of a patient and adjusting treatment for the patient based on comparisons of acquired data and historical data related to the patient. The integrated system includes a central processor that includes a sleep database having baseline sleep information for a generic patient having a similar medical history to the patient. The integrated system also includes an oxygen concentrator or a portable oxygen concentrator (“POC”), a level of consciousness monitor and a mobile communications device. The oxygen concentrator is configured to provide a flow of concentrated oxygen to the patient. The oxygen concentrator is configured to sense and transmit the flow data or flow of air and concentrated oxygen to the patient to the central processor. The oxygen concentrator is specifically configured to sense the flow of the concentrated oxygen that is flowing to the patient so that the central processor is able to analyze the volumetric flow of concentrated oxygen that is being directed to the patient. The oxygen concentrator is in communication with the central processor. The level of consciousness monitor is configured to collect data regarding the patient's state of wakefulness, awareness and alertness. The level of consciousness monitor is in communication with the central processor. The mobile device or tablet computer is configured for transport by the patient. The mobile device or tablet is in communication with the central processor. The central processor collects data from the oxygen concentrator, the level of consciousness monitor and the mobile device and adjusts the oxygen concentrator based on comparisons of the baseline sleep information and the collected data.

In another aspect, the preferred invention is directed to an integrated system for monitoring conditions of a patient including a central processor, an oxygen concentrator configured to provide a flow of concentrated oxygen to the patient, a biometric sensor configured to sense a biometric data point of the patient during use, and a mobile device configured for transport by the patient. The oxygen concentrator is in communication with the central processor. The oxygen concentrator is configured to sense flow data based on the flow of concentrated oxygen during use. The mobile device is in communication with the central processor. The mobile device is configured to sense patient data during use. The central processor is configured to acquire data from the oxygen concentrator, the biometric sensor and the mobile device and adjust the oxygen concentrator based on comparisons of the biometric data point, the patient data and the flow data.

In addition to improving a patient's health by monitoring their sleep quality, a patient's current health and post-operative recovery can be improved by utilizing AI to adjust the volume and concentration of oxygen based on certain existing and anticipated variables related to an oxygen concentrator utilized by a patient.

Patients who have lower blood oxygen levels or general respiratory difficulty or disease are prescribed supplemental oxygen to treat respiratory diseases and conditions that cause a person's blood oxygen saturation level to drop to undesirable levels, such as below eighty-nine percent (89%) or arterial oxygen pressure to fall to undesirable levels, such as below sixty millimeters of mercury (60 mmHg). Reduced blood oxygen levels or arterial oxygen pressure may become low during rest, activity, sleep, at altitude or under numerous other conditions depending on the individual patient and/or the specific conditions or geography the patient is subjected to.

A standard protocol for testing and evaluating exercise capacity and predict outcome in patients with heart failure and other cardiac conditions is the Cardiopulmonary Exercise Test (“CPET” or “CPEX”), also referred to as a VO2 (oxygen consumption) test. CPET or VO2 is a specialized type of stress test or exercise test that measures your exercise ability. Information about the heart and lungs is collected to understand if the body's response to exercise is normal or abnormal. Normal levels of blood oxygen saturation are between ninety-five and one hundred percent (95-100%).

Depending on the disease or conditions that are identified, potentially resulting from the CPT or VO2 tests, the health care provider may prescribe an amount of supplemental oxygen that maintains a patient's blood oxygen level at a certain, desirable level. The American College of Physicians (“ACP”) recommends supplemental long-term oxygen therapy (“LTOT”) in all patients who have severe resting hypoxemia, defined as a partial pressure of oxygen (“PaO₂”) approximately less than or equal to fifty-five millimeters of oxygen (≤55 mmHg) or a peripheral capillary oxygen saturation (“SpO2”) approximately less than or equal to eighty-eight percent (≤88%). The accepted standard of practice is restoration of the SpO2 to a range of approximately eighty-eight to ninety-two percent (88-92%), and there are also British guidelines that make recommendations regarding this range.

These preferred guidelines continue to be ignored by many inexperienced and experienced healthcare providers. Too much oxygen can lead to a dangerous and often fatal condition called hypercapnia.

Administration of high-flow oxygen concentrations has been associated with higher mortality in comparison with a more tailored approach to oxygen therapy. Some data, including those of a randomized controlled trial, provide evidence for the best strategy in patients with an acute exacerbation of chronic obstructive pulmonary disease (“COPD”). These data show that a titrated oxygen administration to achieve an oxygen saturation of between the eighty-eight to ninety-two percent (88-92%) compared with higher saturations results in less respiratory acidosis and better outcome. This is also in accordance with the British Thoracic Society guideline for oxygen therapy in patients with COPD. The preferred oxygen concentrator is configured to sense the flow of concentrated oxygen that is being provided to the patient and transmit the concentrated oxygen flow data to the central processor.

Breathing oxygen at higher than normal partial pressure may lead to hyperoxia and can cause oxygen toxicity or oxygen poisoning. The clinical settings in which oxygen toxicity occurs is predominantly divided into two groups; one in which the patient is exposed to very high concentrations of oxygen for a short duration, and the second where the patient is exposed to lower concentrations of oxygen but for a longer duration. These two cases can result in acute and chronic oxygen toxicity, respectively. The acute toxicity manifests generally with central nervous system (“CNS”) effects, while chronic toxicity has mainly pulmonary effects. Severe cases of oxygen toxicity can lead to cell damage and death.

Carbon dioxide (“CO₂”) is the main stimulus for the respiratory drive in normal physiological states. An increase in carbon dioxide increases the hydrogen ions, which lowers the potential of hydrogen (“pH”). Chemoreceptors are more sensitive to alteration in acid-base balance. An increase in arterial carbon dioxide levels indirectly stimulates central chemoreceptors (medulla oblongata) and directly stimulates peripheral chemoreceptors (carotid bodies and aortic arch). Chemoreceptors are less responsive to oxygen levels. In COPD patients, this effect is blunted as the chemoreceptors develop tolerance to chronically elevated arterial carbon dioxide level. This is when the normal respiratory drive shifts to hypoxic drive and the low oxygen level plays a pivotal role in the stimulation of respiration through the chemoreceptors and maintain respiratory hemostasis. That is why the target pulse oximetry in these patients is eighty-eight to ninety-two percent (88-92%). Thus, it is important not to over oxygenate COPD patients as has been done in the past.

When helping a patient recover post operatively or generally improve their mobility as a result of a lung or heart disease that decreases oxygen flow to the organs, supplemental oxygen is generally provided in three delivery mechanisms: (a) ninety-nine and nine tenths percent (99.9%) pure oxygen from a centralized system in a hospital setting, commonly called “wall oxygen” as it is dispensed through a cannula connected to a valve located on the hospital room wall; (2) metal tanks or cylinders with typically ninety-nine and nine tenths percent (99.9%) pure oxygen; and (3) oxygen concentrators. Oxygen concentrators can be large stationary units generating ten to twenty liters (10-20 L) of oxygen per minute or portable units weighing less than ten pounds (10 lbs) and generating two to three liters of oxygen per minute (2-3 L/min). Oxygen concentrators take ordinary air that is approximately twenty percent (20%) oxygen, seventy-nine percent (79%) Nitrogen and one percent (1%) trace gases and concentrates the oxygen through processes known as pressure swing adsorption (“PSA”) or vacuum swing adsorption (“VSA”). Efficient PSA or VSA systems can generate approximately ninety-five to ninety-six percent (95-96%) pure oxygen.

An important requirement for improved post-operative recovery and improved health is exercise and mobility. Exercise training is widely regarded as the cornerstone of pulmonary rehabilitation in patients with COPD. Indeed, exercise training has been identified as the best available means of improving muscle function and exercise tolerance in patients with COPD. So, exercise training truly makes a difference in the life of patients with COPD.

In order to increase mobility in patients requiring supplemental oxygen, decreasing the weight of the supplemental oxygen source and increasing the mobility of the supplemental oxygen source has been a focus of many respiratory specialists. In addition to decreasing the weight of the supplemental oxygen source, there has also been a focus on mobility and duration of the supplemental oxygen source. Oxygen cylinders, for example, have a finite source of oxygen and are very heavy, particularly for a patient with respiratory problems. When the supply of oxygen is depleted, a user must change to another cylinder. Weight and size of the cylinders is directly correlated to length of oxygen supply. For oxygen concentrators that are portable and operate on batteries 60, the battery run time is a focus to extend the running time of the portable oxygen concentrator. Generating enriched oxygen requires power from the battery 60 or other power source. Bigger batteries 60, such as the second battery 60b, will supply a longer run time, but will make the portable oxygen concentrator 14 weigh more, thereby directly impacting oxygen usage due to the heavier weight. The significant benefit of the POC 14 over a cylinder of purified oxygen is that the POC 14 generates oxygen continuously as long as it is being powered. If the battery 60 of the POC 14 runs out of power, a fresh battery 60 can be installed, the POC 14 can be plugged into a standard outlet or the POC 14 can be otherwise powered to run the compressor. When a cylinder runs out of oxygen, the user must have a replacement nearby, otherwise they do not have a source of supplemental oxygen.

An improvement to these systems is to more efficiently and effectively oxygenate patients with the POC to extend the lifetime of the power source, which is typically a battery. Pulse dose oxygen delivery systems for POC's were developed approximately twenty (20) years ago to improve the efficiency of POC. Instead of oxygen flowing continuously, oxygen in a pulse dose system delivers a bolus oxygen when it senses the beginning of a patient's inhalation through a nasal cannula. The preferred POC is able to sense both the continuous and pulse dose concentrated oxygen flow and transmit this data to the central processor. Because the typical breathing cycle involves inspiration about one-third (⅓) of the time and exhalation the other two-thirds (⅔) of the breathing cycle, pulse dose oxygen delivery significantly increases the efficiency of the oxygen delivery process. However, additional improvements can be made to improve the power usage of the POC to make the battery last longer and produce a more accurate and effective delivery of the oxygen to improve a patient's recovery and health condition.

Numerous factors impact the efficiency of a POC. For example, an efficient POC will generate ninety-five percent (95%) pure oxygen. POC's however, lose their efficiency over time as the zeolites used in the PSA or VSA process become contaminated with water, Nitrogen or other contaminants and break down, thereby reducing their efficiency in concentrating oxygen and ultimately lowering the purity of the oxygen produced during the POC operating cycles. Supplemental oxygen is considered a drug in the US. Patients must have a prescription in order to purchase a POC. POC's, to be cleared by the US Food and Drug Administration (“FDA”) for marketing and sale, must produce at least eighty-two percent (82%) concentrated oxygen.

In addition to different levels of oxygen purity, pulse dose POC's provide different levels or quantities of oxygen at various breathing rates. Depending on the breathing rate of the patient, a different amount of oxygen is delivered to the patient. For example, a POC that is set to deliver six hundred milliliters (600 mL) of pulse dose oxygen over a one minute period may deliver a forty milliliter (40 mL) bolus oxygen at fifteen (15) breaths per minute, a thirty milliliter (30 mL) bolus of oxygen at twenty (20) breaths per minute and a fifteen milliliter (15 mL) bolus of oxygen at forty (40) breaths per minute. Some POC's deliver the same size bolus of oxygen at all breathing rates. This method can result in a patient having significantly different amounts of oxygen delivered based on their breathing rates.

Another factor when considering operation of the POC and delivery of the appropriate amount of oxygen to the patient is the altitude environment where the POC is operating. Altitude impacts both a patient's intake of purified oxygen and a POC's oxygen delivery quantity. Although the percentage of oxygen in inspired air is constant at different altitudes, the fall in atmospheric pressure at higher altitude decreases the partial pressure of inspired oxygen and hence the driving pressure for gas exchange in the lungs. An ocean of air is present up to nine to ten thousand meters (9,000-10,000 m), where the troposphere ends, and the stratosphere begins. The weight of air above us is responsible for the atmospheric pressure, which is normally about one hundred Kilopascals (100 kPa) at sea level. This atmospheric pressure is the sum of the partial pressures of the constituent gases, including oxygen and nitrogen, and also the partial pressure of water vapor (6.3 kPa at 37° C.). As oxygen is twenty-one percent (21%) of dry air, the inspired oxygen pressure is approximately 0.21×(100−6.3)=19.6 kPa at sea level.

Atmospheric pressure and inspired oxygen pressure fall roughly linearly with altitude to be fifty percent (50%) of the sea level value at five thousand five hundred meters (5,500 m) and only thirty percent (30%) of the sea level value at eight thousand nine hundred meters (8,900 m) (the height of the summit of Mount Everest)). A fall in inspired oxygen pressure reduces the driving pressure for gas exchange in the lungs and in turn produces a cascade of effects right down to the level of the mitochondria, the final destination of the oxygen.

People with COPD or recovering post-operatively are not typically climbing high mountains, but even in cities like Boulder, Colorado the effective oxygen percentage drops to seventeen and three tenths percent (17.3%) and Flagstaff, Arizona has an effective oxygen percentage of sixteen percent (16%). That is four (4) basis points or twenty percent (20%) less oxygen than at sea level.

Because POC and PSA systems utilize a compressor to create a pressurized chamber in the sieve column in order to create an adsorption process between the zeolite and Nitrogen in ambient air, the reduced atmospheric pressure requires the compressor to work harder to generate the same amount of pressure when operating and increased altitudes. Compressor output can decrease up to thirty-six percent (36%) at altitudes of eight thousand feet (8,000 ft) above sea level. One way to overcome this limitation of known POC and PSA systems and, specifically, their control systems, is to increase the revolutions per minute (“RPM”) of the compressor such that a greater volume of air per minute is pushed through the compressor.

Integrating AI into the control system of the POC and PSA systems to adjust the operation of the system due to various environmental and system factors and variables is desirable and the preferred embodiment of the present invention addresses certain of the shortcomings of know PSA and POC systems and their controls.

### BRIEF DESCRIPTION OF THE SEVERAL VIEWS OF THE DRAWINGS

The foregoing summary, as well as the following detailed description of preferred embodiments of the system, mechanism and method of the preferred present invention, will be better understood when read in conjunction with the appended drawings. For the purposes of illustrating the preferred system and method for improving postoperative patient recovery with at least an oxygen concentrator, there are shown in the drawings preferred embodiments. It should be understood, however, that the application is not limited to the precise arrangements and instrumentalities shown. In the drawings:

FIG. 1 is a front perspective view of an integrated system and related components in accordance with a preferred embodiment of the present invention;

FIG. 2 is a front elevational view of a preferred mobile device of the system of FIG. 1;

FIG. 3 is a view of a sleep chart or sleep pattern that may be utilized with the system of FIG. 1;

FIG. 4 is a schematic diagram of components of the integrated system and interaction between the components of the integrated system of FIG. 1;

FIG. 5 is a schematic diagram of components of the integrated system and interaction between the components of the integrated system of FIG. 1 with examples of stored and acquired data associated with various components of the preferred system;

FIG. 6 is a side elevational view of components of the integrated system of FIG. 1;

FIG. 7 is a rear perspective view of a portable oxygen concentrator of the integrated system of FIG. 1, wherein an adsorbent container, compressor and flow path through the portable oxygen concentrator are shown within a housing of the portable oxygen concentrator;

FIG. 8 is a schematic diagram of components of the integrated system of FIG. 1, including the portable oxygen concentrator and variables that may be considered when operating the portable oxygen concentrator under different conditions or based on data collected from operation of the portable oxygen concentrator;

FIG. 9 is another schematic diagram of components of the integrated system of FIG. 1, including the portable oxygen concentrator and various sensors or acquired data that may be considered when operating the portable oxygen concentrator under different conditions or based on data collected from operation of the portable oxygen concentrator;

FIG. 10 is front elevational view of a display of a mobile device related to connecting the integrated system of FIG. 1 with sensor or other devices; and

FIG. 11 is a front elevational view of a display of a mobile device related to monitoring a portable oxygen concentrator of the integrated system of FIG. 1.

### DETAILED DESCRIPTION OF THE INVENTION

Certain terminology is used in the following description for convenience only and is not limiting. Unless specifically set forth herein, the terms “a”, “an” and “the” are not limited to one element but instead should be read as meaning “at least one”. The words “right,” “left,” “lower” and “upper” designate directions in the drawings to which reference is made. The words “inwardly” or “distally” and “outwardly” or “proximally” refer to directions toward and away from, respectively, the patient's body, or the geometric center of the preferred system and related parts thereof. The words, “anterior”, “posterior”, “superior,” “inferior”, “lateral” and related words and/or phrases designate preferred positions, directions and/or orientations in the human body to which reference is made and are not meant to be limiting. The terminology includes the above-listed words, derivatives thereof and words of similar import.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

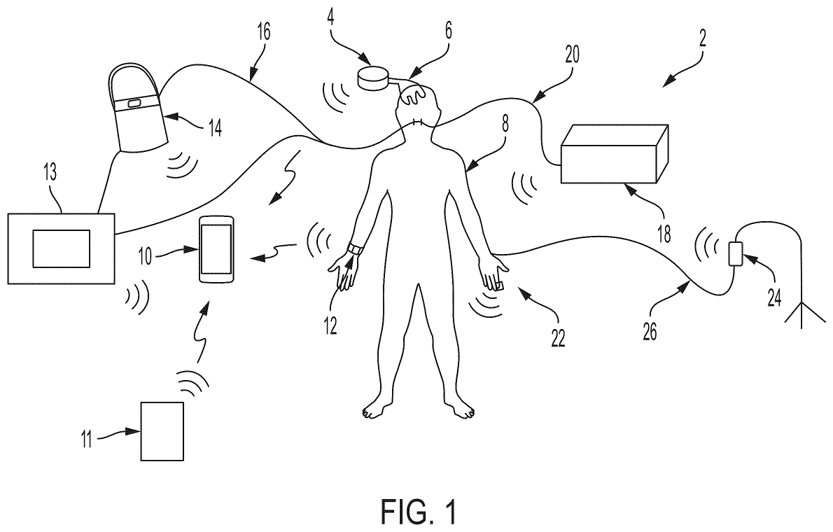

Referring to FIGS. 1-5, an integrated system, generally designated 2, constructed in accordance with a preferred embodiment of the present invention includes a level of consciousness (“LOC”) monitor 4 and a consciousness sensor 6. Distal ends of the consciousness sensor 6 are preferably removably secured to the forehead of an individual or patient 8 via biocompatible adhesive material. Examples of the consciousness sensor 6 that have been previously sold include sensors for the Snap II Level of Consciousness Monitor distributed by Stryker Corporation and as further described and claimed in U.S. Pat. No. D541,421, which is incorporated herein by reference in its entirety. The consciousness sensors 6 detect and receive electroencephalogram (“EEG”) signals from a person's or patient's 8 brain. Removably attached to the sensor 6 at the proximal end is the LOC monitor 4. The analog EEG signals are preferably transmitted through the consciousness sensor 6 to the LOC monitor 4.

The LOC monitor 4 is an improvement over Snap II, wherein the Snap II processor, battery, display, memory and other electronics were contained in a single monitor. The LOC monitor 4 receives an analog EEG signal from the consciousness sensor 6, converts the signal into a digital signal, processes the EEG signal as described in U.S. Pat. No. 5,813,993 and US Patent Application Publication Nos. 2007/0167694, titled, “Integrated Portable Anesthesia and Sedation Monitoring Apparatus” and 2016/0066806, titled, “Impedance Bootstrap Circuit for an Interface of a Monitoring Device,” each of which is incorporated herein by reference in their entirety. The processed EEG, referred herein as a Snap Index, is transferred via either a tethered connection such as a USB cable or wirelessly, such as via Bluetooth to a phone, notepad, tablet, laptop or desktop computer 10. The tablet 10 has the ability to receive and process the Snap data as would be understood by one of skill in the art upon review of the present disclosure.

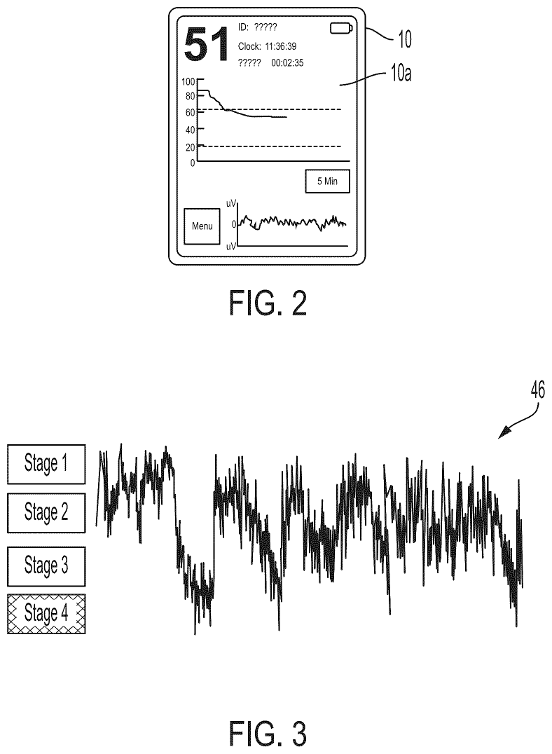

The tablet 10 preferably has the ability to receive and operate a software application 28 designed to display the Snap data in several different modes. Wherein the original Snap II device was only capable of displaying the Snap Index in a mode that measured the level of consciousness of a person under sedation, the software application 28 provided on the tablet 10 is able to display several modes for different diagnostic purposes. Whereby, when the LOC monitor 4 is used in a surgery on a patient under anesthesia, the tablet 10 displays a graph showing the patient's Snap Index to reflect their level of consciousness, as further shown in FIG. 2, wherein an example graph of the patient's Snap Index is shown on a display 10a of the tablet 10.

In a second mode, or second preferred software application 30, the tablet 10 could also display a chart showing the sleep state of a patient, as shown in FIG. 3. In this second mode, the Snap Index is converted to a graph reflecting whether a patient is in Stage 1, Stage 2, Stage 3 (also Stage 4) or rapid eye movement (“REM”) sleep. It is anticipated that other software applications could be installed on the tablet 10 that would display the processed EEG signals in a form, such as, for example, to reflect a meditation state or non-rapid eye movement (“NREM”) v. REM.

A smart watch, fitness or health tracker device 12, such as a health watch 12, including, the Fitbit Versa, Suunto 3, Apple Watch, Polar M430 and the Samsung Gear Sport, may be incorporated into the integrated health monitoring system 2. The health watch 12 can transmit one or more, among other things, biometric data or patient data including pulse/heart rate, body temperature, location, calories burned, distance traveled, time, steps, race, age, sex or mobility. Typically, a software application associated with the health watch 12 is downloaded to a user's phone, tablet or other computer 10. For purposes of the preferred invention described herein, a software application 32 associated with the health watch 12 is preferably installed on the tablet 10.

In the preferred embodiment, a mobile device, which may be comprised of the smart watch, fitness or health tracker device 12, the phone, notepad, tablet, laptop or desktop computer 10 or other mobile device associated with the patient, is configured for transport by the patient. The mobile device 10 is in communication with a central processor 42 to detect, transmit, acquire and sense variables or data associated with the patient during use.

A concentrated oxygen source or POC 14 is also preferably incorporated into the integrated health monitoring system 2. The POC 14 is configured to provide a flow of concentrated oxygen to the patient, during use. The POC 14 is in communication with a central processor 42 of the preferred system 2. The POC 14 is configured to sense flow data based on the flow of concentrated oxygen from the POC 14 to the patient during use. The concentrated oxygen source or portable oxygen concentrator 14 generally may include or comprise oxygen tanks, stationary oxygen concentrators and portable oxygen concentrators. In the preferred embodiment, concentrated oxygen is delivered from the concentrated oxygen source 14, such as the POC 14. Sample POC's include the POC's described in U.S. Pat. No. 9,199,055, titled “Ultra Rapid Cycle Portable Oxygen Concentrator;” U.S. Pat. No. 8,888,902, titled, “Portable Oxygen Enrichment Device and Method of Use” and D889,394, titled “Battery” and International Patent Application Publication Nos. WO 2018/226532 (“WO-532”), titled “Configurable Oxygen Concentrator and Related Method” and 2017/165749, titled, “Positive Airway Pressure System with Integrated Oxygen,” (collectively “POC Patents”) each of which is incorporated herein by reference in their entirety. In the preferred embodiment, the POC 14 has a means for transmitting data and receiving data and instructions either wired/tethered or wirelessly such that a user of the tablet 10 can view information on a software application 34 on the tablet 10 that was transmitted by the POC 14 to the tablet 10. In addition, in an alternative preferred embodiment, the user of the tablet 10 can view information received from the POC 14 and send instructions to the POC 14 to remotely operate all or some functions of the POC 14 via a software application 34.

The POC 14 preferably delivers concentrated oxygen to users needing or desiring higher concentration levels of oxygen. Typical air is approximately seventy-nine percent (79%) Nitrogen, twenty percent (20%) Oxygen and one percent (1%) Argon and other gases at sea level. The POC's 14 of the preferred embodiment typically supply eighty-five to ninety-five percent (85-95%) pure Oxygen. Users and patients 8, for example, that suffer from chronic obstructive pulmonary disease (COPD) are regularly prescribed supplemental oxygen. Other potential users or patient's 8 of supplemental concentrated oxygen include people suffering from asthma, bronchitis, cystic fibrosis and other respiratory diseases preventing proper pulmonary oxygen carbon dioxide exchange such that a person's blood oxygen level drops below a medically acceptable level.

It is further contemplated that the POC 14 could be connected to a ventilator such as the AVEA ventilation system by Vyaire or a high flow nasal cannula system, wherein the ventilator and high flow nasal cannula system are identified generically by reference number 13, such as those provided by Vapotherm or TNI SoftFlow System. The ventilator and high flow nasal cannula systems 13 are not limiting and the POC 14 may be connected to and utilized with nearly any variety of ventilator or high flow nasal cannula that is utilized to provide air or breathing assistance to the patient. Connecting the POC 14 to the ventilator or high flow nasal cannula 13 is preferably utilized to increase the concentrated oxygen levels provided by the ventilators or high flow nasal cannulas 13 while potentially being able to reduce the volume of air being delivered to the patient, thereby making the ventilator or nasal cannula 13 more comfortable to a patient. The primary benefit of ventilators and high flow nasal cannulas 13 is their high volumetric flow rate of air to the patient, typically fifty to sixty liters per minute (50-60 LPM), to wash out dead space in a patient suffering from acute respiratory distress. Wiping out the dead space clears the accumulated carbon dioxide from the respiratory tract, thereby pushing more air and oxygen to the lungs through evacuation of the carbon dioxide. For those patients having both difficulty inhaling and exhaling and sufficient oxygen and carbon dioxide exchange, titrating additional oxygen into the ventilator or high flow nasal cannula 13 is beneficial. The POC 14 is, therefore, able to enhance or increase the oxygen level in the air introduced to the patient through the ventilator or the high flow nasal cannula 13 by introducing the concentrated oxygen from the POC 14 to the ventilator or the high flow nasal cannula 13. The ventilator or high flow nasal cannula system 13 and the POC 14 are both preferably in communication with the tablet 10 to control and direct the introduction of purified oxygen from the POC 14 to the ventilator or high flow nasal cannula system 13 to enhance operation of the ventilator or high flow nasal cannula system 13. The concentrated oxygen produced by the POC 14 is mixed with the air driven by the ventilator or high flow nasal cannula system 13 to the patient, such that higher levels of concentrated oxygen are present in the air introduced to the patient from the ventilator or high flow nasal cannula system 13.

Patient safety, security and privacy are critical when considering the transmission of patient and equipment data, particularly a patient's health information or medical records. Depending on the level of information and remote control desired by the user of the POC 14, the software application 34 can be written with certain security levels and protocols such that a user of the tablet 10 is limited by the security level established by the user of the POC 14. For example, a lower security level can be established to allow limited access to information such as the remaining power level or the power level of the POC's 14 battery 60 or the oxygen flow rate level. A medium level of security may allow the user of the tablet 10 to see additional information related to oxygen usage, such as the location of the POC 14, which may be determined by a global positioning system (“GPS”) or related sensors integrated into the POC 14. The highest level of security could include the ability of a user of the tablet 10 to send instructions to the POC 14 to change the oxygen delivery level or power the POC 14 off or on. If the POC 14 includes a configuration that delivers medication, the security level could include the ability to manage the level of medication delivered via the POC 14. An oxygen cannula 16 is also preferably incorporated into the integrated system 2, thereby creating a channel for concentrated oxygen to travel from the POC 14 to patient 8. The oxygen cannula 16 may also be utilized with the ventilator or high flow nasal cannula system 13 to provide oxygen to the patient 8.

The preferred POC 14 may include several differently sized batteries 60, such as a first battery 60a that has a lower electrical storage capacity and a lower weight than a second battery 60b. The POC 14 may have additional differently sized batteries 60, but the first and second batteries 60a, 60b are shown and described for efficiency herein. The first battery 60a may be utilized for portability when the user does not intend to be away from a direct electrical connection or power source for an extended period of time and the second battery 60b may be utilized when the user intends to be away from a recharging station or direct electrical connection for longer periods of time. The POC 14 is preferably configured to track the power levels of the batteries 60, 60a, 60b at the central processor 42 and alert the user when power is becoming low. The AI of the central processor 42 may adjust the time when a warning is provided to the user based on location data indicating that the user is distanced from recharging capability or a direct power connection, such as when travelling or based on the specific user's history related to the time it takes for the user to replace or recharge the battery 60, 60a, 60b after a warning is initially provided. The patient data may include data including oxygen purity, oxygen bolus size, battery power, remaining battery power, battery power consumption, patient breathing rate, compressor revolutions per minute or alarm codes. The patient data may also include a level of consciousness of the patient, patient heart rate, patient blood pressure, patient body temperature, patient blood oxygen saturation, patient tidal volume and patient lung volume, which are sensed and transmitted by appropriate sensors that are in communication with the central processor 42.

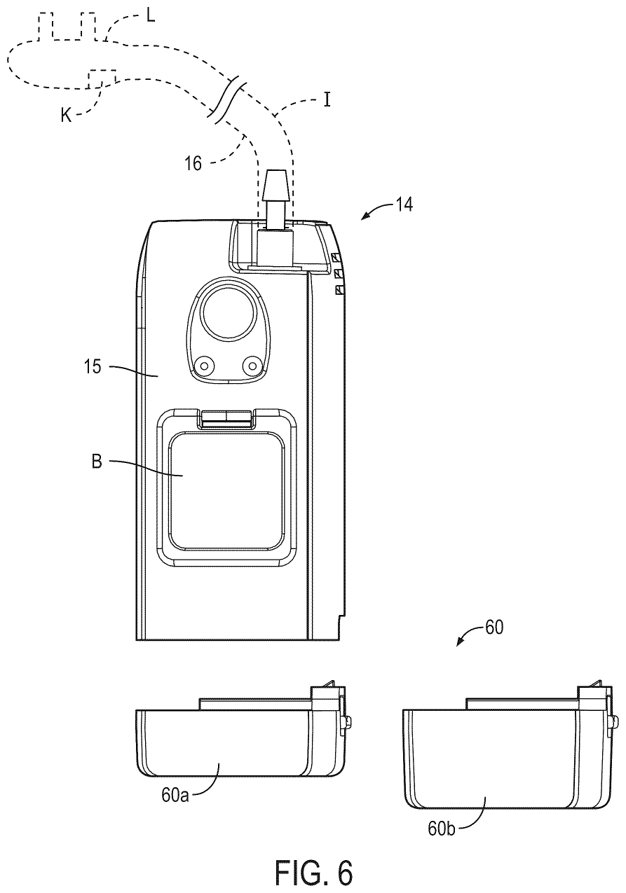

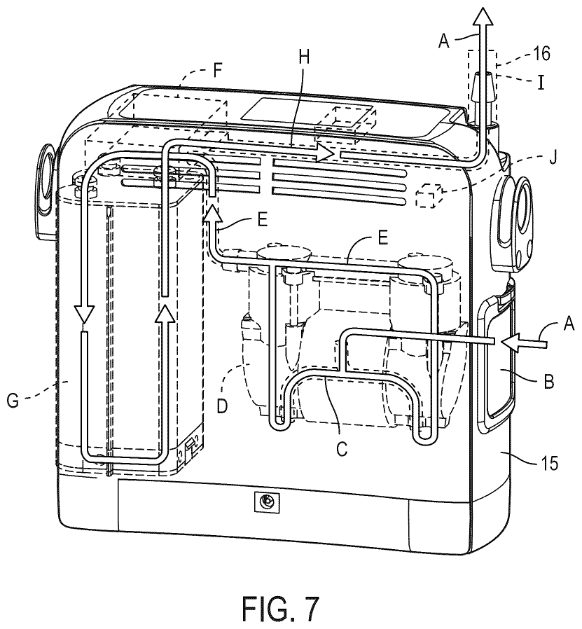

The preferred POC 14 is described in detail in the POC Patents and is generally shown in FIGS. 6 and 7 herein. The POC 14 of the preferred embodiment has a gas pathway A that the air and concentrated oxygen follows through the POC 14 and ultimately to the patient. The gas pathway A is preferably initially guided by an air inlet B where ambient air flows into a housing 15 of the POC 14 and for receiving ambient air. The inlet B is connected to a conduit C to carry the ambient air into a compressor D. The ambient air is compressed in the compressor D and then expelled into a second conduit E that transfers the compressed air to a manifold F. The manifold F controls the flow of compressed air from the second conduit E into a molecular sieve assembly G. Nitrogen is preferably separated from oxygen in a molecular sieve assembly G by a pressure swing adsorption process. The concentrated oxygen produced from the pressure swing adsorption process in the molecular sieve assembly G flows out of molecular sieve assembly G and into a third conduit H. The concentrated oxygen flows through the third conduit H and out through a cannula connector I, which delivers the concentrated oxygen to the patient.

An oxygen concentration level sensor J is preferably located downstream of the sieve assembly G in the third conduit H or otherwise in the flow of concentrated oxygen. The oxygen concentration level sensor J measures or collects data regarding the percentage of oxygen in the gas, preferably concentrated oxygen, flowing in the third conduit H. FDA cleared oxygen concentrators or POC's 14 must generate at least eighty-two percent (82%) concentrated oxygen before an alarm is required to notify users of low oxygen concentration.

Current oxygen delivery strategies for an oxygen concentrator involve the delivery of a flow of oxygen in either pulse dose boluses or continuous flow. Continuous flow may be limited to a specific volume of oxygen flow, such as three liters per minute (3 Lpm). Larger flow rates require larger compressors weighing over fifteen pounds (15 lbs) making the resulting oxygen concentrator generally not portable or at least not practically portable for a typical patient with respiratory issues. Many patients do not need a continuous flow of concentrated oxygen at all times. Pulse dose units came about to improve the efficiency of concentrated oxygen delivery to patients, resulting in a more efficient delivery of the concentrated oxygen to the patient. The pulse dose POC 14 of the preferred embodiment may be designed to only deliver a bolus of oxygen when a negative pressure sensor K, located downstream of the modular sieve assembly G, senses the beginning of a patient's inhalation through a nasal cannula L connected to the cannula connector I. A predetermined bolus of oxygen is released from the POC 14 to the patient after the negative pressure sensor K senses the inhalation.

In the preferred embodiment, the system 2 includes a biometric sensor configured to sense a biometric data point of the patient during use. The biometric sensor may be comprised of numerous sensors associated with numerous devices that are designed and configured to acquire data and transmit the data to the central processor 42. The biometric sensors may include, but are not limited to, the pulse oximeter 22, the negative pressure sensor K, the consciousness sensor 6 of the LOC monitor 4, a temperature sensor, a blood pressure sensor, a heart rate monitor, a pulse sensor, and other related biometric sensors that may be able to detect, acquire and transmit biometric information related to the patient. The consciousness sensor 6 preferably detects, acquires and transmits electrical activity in the patient's brain and the central processor 42 is preferably able to determine the patient's level of consciousness based on the sensed electrical activity.

Pulse dose POC's typically deliver a bolus of oxygen based on a preset breathing rate and flow volume, without taking into account the literal breathing rate of the patient. The POC may, for example, have preset volumes of two hundred milliliters per minute (200 mL/min), four hundred milliliters per minute (400 mL/min), six hundred milliliters per minute (600 mL/min and eight hundred milliliters per minute (800 mL/min). Preset breathing rates per International Organization for Standardization (“ISO”) standards for oxygen concentrators are fifteen, twenty, twenty-five, thirty, thirty-five and forty (15, 20, 25, 30, 35 and 40) breaths per minute (“BPM”). At a setting of two hundred milliliters per minute (200 mL/min), a patient breathing at fifteen BPM (15 BPM) would receive a thirteen and three tenths milliliter (13.3 ml) bolus of oxygen. At forty breaths per minute (40 BPM), the bolus would be five milliliters (5 mL). There are a few POC's that deliver the same bolus at each setting. This is very confusing to patients and care givers because, for example, at a breathing rate of fifteen beats per minute (15 BPM) the POC that is delivering a set volume of thirty milliliters (30 mL) is delivering four hundred fifty milliliters per minute (450 mL/min) while at a rapid breathing rate of forty breaths per minute (40 BPM), the POC is delivering one thousand two hundred milliliters (1200 mL) of concentrated oxygen. Such a system wastes concentrated oxygen, is not operating efficiently and may be over oxygenating the patient.

As further described herein, the pulse oximeter 22 is used to measure the blood oxygen saturation level or SpO2. A normal range of SpO2 for healthy individuals is approximately ninety-five to one hundred percent (95-100%) SpO2. For COPD patients having SpO2 levels that consistently fall below eighty-eight percent (88%), supplemental oxygen is prescribed. For other patients with cardiac or peripheral vascular illnesses, supplemental oxygen is often prescribed when saturation levels drop below ninety-three percent (93%). When a respiratory therapist or pulmonologist has evaluated the patient and determined supplemental oxygen is needed to increase the patient's SpO2 levels, the respiratory therapist, pulmonologist or other medical professional needs to determine the correct fraction of inspired oxygen (FiO2) to provide to the patient. At sea level the FiO2 of ordinary air is approximately twenty percent (20%). FiO2 is an estimate of the oxygen content a person inhales.

When prescribing an FiO2 currently, there is a significant amount of educated guessing by the respiratory therapist, pulmonologist or other medical professional. General tables have been created to reflect the typical FiO2 for various oxygen delivery devices such as nasal cannulas L, venturi masks and high flow nasal cannulas. For example, a nasal cannula L set at a one liter per minute (1 L/min) flow rate can increase FiO2 to twenty-four percent (24%), a two liter per minute (2 L/min) flow rate can increase FIO2 to twenty-eight percent (28%), a three liter per minute (3 L/min) flow rate can increase FIO2 to thirty-two percent (32%), a four liter per minute (4 L/min) flow rate can increase FIO2 to thirty-six percent (36%), a five liter per minute (5 L/min) flow rate can increase FIO2 to forty percent (40%), and a six liter per minute (6 L/min) flow rate can increase FIO2 to forty-four percent (44%). Typically, POC users utilize the nasal cannula L for delivery of the concentrated oxygen to the nasal cavity of the patient. These FiO2 estimates can vary due to many factors with many studies showing the oxygen actually delivered is less than reflected in the tables. The inaccuracy of oxygen delivery results in oxygen and power waste, inefficient operation of the POC and, potentially, either not enough oxygen, hypoxia or too much oxygen, hypoventilation.

One method to improve the delivery of supplemental oxygen is to utilize a spirometer in the establishment of a recommended FiO2. The spirometer is used to measure tidal volume, inspiratory reserve volume, and expiratory reserve volume. Tidal volume is the amount of air that moves in or out of the lungs with each respiratory cycle. Tidal volume measures around five hundred milliliters (500 mL) in an average healthy adult male and approximately four hundred milliliters (400 mL) in a healthy adult female.

The general table above was calculated based on the above average tidal volumes. Assuming wall oxygen from a dedicated oxygen supply is approximately ninety-nine and ninety-nine hundredths percent (99.99%) pure oxygen, the FiO2 is calculated for a nasal cannula as follows—an average male patient inspires five hundred milliliters (500 mL) of ambient air at sea level while simultaneously inhaling a bolus of ninety-nine and ninety-nine hundredths percent (99.99%) oxygen. At continuous flow of one liter per minute (1 L/min) of wall oxygen the FiO2 is approximately twenty-three percent (23%) at a breathing rate of fifteen breaths per minute (15 BPM). For an adult female, her FiO2 would be approximately twenty-four percent (24%). At fifteen breaths per minute (15 BPM) average inspiratory time is one and thirty-three hundredths seconds (1.33 sec) and actual oxygen delivered to the alveoli is approximately sixty percent (60%) of the inspiratory time or about eighty hundredths of a second (0.80 sec). Tidal volume is typically seven milliliters per kilogram (7 mL/kg) of ideal body weight.

FiO2 can be readily adjusted for a person at rest with a steady breathing rate that is connected to wall or cylinder oxygen with approximately ninety-nine and ninety-nine hundredths percent (99.99%) purity. With numerous changing variables impacting the oxygen delivery of POC's, it has been difficult for the practitioner and patient to adjust oxygen settings to meet FiO2 requirements that meet SpO2 goals.

Referring to FIG. 1, the POC 14 is utilized as part of an overall patient health monitoring system 2. Referring to FIGS. 6 and 7, the control, operation and maintenance of the POC 14 of the patient health monitoring system 2 may be directed and optimized by the AI and machine learning of the system 2. Variables that have an impact on patient FiO2 include oxygen purity, sieve module or oxygen purity degradation, volume of oxygen delivered, duration of oxygen delivered, type of oxygen delivery such as if the concentrated oxygen is delivered as a bolus in response to an inhalation signal, altitude, patient motion, tidal volume and other related variables and factors. In addition, studies have shown that tidal volume increases with exercise in that the body requires more oxygen to fuel its cells when the patient is exercising.

Prior art devices that tie increasing oxygen flow to readings on a pulse oximeter fail to take into account many of the listed POC variables. The preferred POC 14 described herein takes into account POC variables in addition to other factors to provide a more accurate FiO2 than known in the prior art.

A first variable to contemplate when considering how to set oxygen flow for the patient from the POC 14 is oxygen purity. FDA cleared POC's typically operate between eighty-two and ninety-five percent (82-95%) purity of the delivered concentrated oxygen. POC's typically do not generate one hundred percent (100%) oxygen purity because the zeolites used in the molecular sieve G only separates and removes Nitrogen from the ambient air, not additional elements. Trace gases such as Argon, Neon and carbon dioxide (CO₂) pass through the zeolite bed of the molecular sieve G with the oxygen molecules. It is possible to separate or otherwise remove these trace gases, but the additional filtering adds weight and cost to the POC 14, which is lightweight and portable, and the less than close to one hundred percent (100%) purity of the concentrated oxygen from the POC 14 is typically sufficient to provide the desired therapy to the patient.

Even from the beginning, therefore, the POC 14 provides a different FiO2 than a cylinder of purified oxygen with an approximately ninety-nine and ninety-nine hundredths percent (99.99%) purity oxygen at the same flow rate. This is not a negative aspect from a clinical standpoint, as the POC 14 may be adjusted when determining the intended FiO2 and subsequent SpO2 target. The FiO2 and the source of the concentrated oxygen is a factor that is preferably considered and accounted for by the system 2 when treating the patient.

A second variable for consideration that impacts FiO2 of the POC 14 is degradation in performance of molecular sieve material in the molecular sieve G. Because the POC 14 takes ambient air into the gas pathway A and then concentrates the air into enriched or concentrated oxygen, the POC 14 draws in moisture from the ambient air into the molecular sieve G. If there is no drying mechanism or moisture wicking apparatus as part of the gas pathway A at the inlet B or otherwise positioned before entry into the molecular sieve G, the moisture travels to and is often trapped in the molecular sieve G. Even with a drying mechanism or a moisture wicking apparatus, moisture may enter and become trapped in the zeolite material of the molecular sieve G after prolonged use. Zeolites contained in the molecular sieve G for concentrating the ambient air into the concentrated oxygen rapidly absorbs moisture. The absorption of water into the zeolite material reduces the surface area of the zeolites for bonding to Nitrogen during the PSA process, thereby reducing the efficiency and capacity of the molecular sieve G. In more humid environments, the degradation of oxygen purity resulting from the contamination of the zeolite material increases more quickly. In addition, increasing the flow setting on the POC 14 requires the POC 14 to process more air to generate more concentrated oxygen. The increased flow of humid, ambient air brings more moisture into the molecular sieve G, thereby further accelerating the degradation of the zeolite material. Replacing the sieve modules G, such as is described in U.S. Pat. Nos. 9,199,055 and 10,507,300, as well as International Patent Application Publication No. WO 2018/226532, results in a new oxygen purity when the POC 14 is operating in the same environment and under the same conditions, further supporting the need for an improved method for delivering accurate FiO2 with the POC 14 utilizing data acquisition, prediction and AI. The preferred system 2 is able to measure and control the POC 14, as well as predict molecular sieve G performance and expected durations for a useful life of the molecular sieve G based on tracking the variables and historical performance of the POC 14.

Another variable for consideration that impacts FiO2 of the POC 14 is a testing or validation parameter. For example, POC's cleared by the FDA must adhere to International Standards 80601-2-67 and 80601-2-69 relating oxygen flow settings and measurement validation. Under these ISO standards, an applicant for 510 k clearance from the FDA of a POC that delivers pulse dose oxygen must provide a table reflecting the oxygen bolus sizes delivered at all settings for breathing rates of fifteen, twenty, twenty-five, thirty, thirty-five and forty breaths per minute (15, 20, 25, 30, 35 and 40 BPM). Per the standards, the stated bolus volumes may have a plus or minus fifteen percent (±15%) variance from the volume stated in the table. Certain five pound (5 lb) POCs have five (5) settings that state in their table that the POC provides two hundred milliliters per minute (200 mL/min) at setting number one (1), four hundred milliliters per minute (400 mL/min) at setting number two (2), six hundred milliliters per minute (600 mL/min) at setting number three (3), eight hundred milliliters per minute (800 mL/min) at setting number four (4) and one thousand milliliters per minute (1,000 mL/min) at setting number five (5), respectively. Further breaking down setting number three (3), bolus volumes at each breathing rate would be as follows: forty milliliters (40 mL) at fifteen breaths per minute (15 BPM), thirty milliliters (30 mL) at twenty breaths per minute (20 BPM), twenty-four milliliters (24 mL) at twenty-five breaths per minute (25 BPM), twenty milliliters (20 mL) at thirty breaths per minute (30 BPM), seventeen and fourteen hundredths milliliters (17.14 mL) at thirty-five breaths per minute (35 BPM) and fifteen milliliters (15 mL) at forty breaths per minute (40 BPM). Applying the standard's variability at fifteen breaths per minute (15 BPM) at setting number three (3), the actual bolus volume of enriched oxygen delivered by the POC can be thirty-four milliliters (34 mL) to forty-six milliliters (46 ml). The oxygen purity, however, further complicates matters as it can range from eighty-two to ninety-five percent (82-95%). Applying this range of acceptable concentrations, the actual bolus of oxygen delivered may range from twenty-seven and nine tenths milliliters (27.9 mL) to forty-four and sixteen hundredths milliliters (44.16 mL) at fifteen breaths per minute (15 BPM) on setting number three (3). This results in an FiO2 range between twenty-six and one-half percent (26.5%) and twenty-nine and seven tenths percent (29.7%) or, roughly, a variability of one liter per minute (1 L/min) of pure oxygen.

A benefit of the preferred POC 14 over oxygen cylinders, besides its ability to continuously provide enriched oxygen over time, is the ability to travel with the POC 14, such as by travelling on an aircraft with the POC 14. Prior art oxygen cylinders, being pressurized oxygen, are banned from commercial aircraft for safety reasons. The POC 14, however, because it provides enriched oxygen by concentrating the ambient air, such as ambient air in the aircraft cabin, have altitude limitations, but are able to operate in an aircraft during flight. Although oxygen concentration does not change with increased altitude, barometric pressure decreases causing nitrogen and oxygen particles to spread apart. The patient's body has to take in more air in order to achieve the same FiO2 as at sea level. The equivalent FiO2 in Boulder, Colorado at an elevation of five thousand four hundred thirty feet (5,430 ft) is approximately seventeen and three tenths percent (17.3%) and in Aspen, at eight thousand feet (8,000 ft) the FiO2 is approximately fifteen and four tenths percent (15.4%). The compressor D of the POC 14 will have to either increase its RPM slightly to increase the amount of air being compressed to maintain the same purity compared to at sea level or stay at the same revolutions per minute level and generate oxygen at a lower purity. Most POC's are rated to ten thousand feet (10,000 ft) of altitude, meaning they will still generate at least eighty-two percent (82%) enriched oxygen at that elevation.

Air travel can create challenges for patients on supplemental oxygen and even those that do not need supplemental oxygen at sea level. Aircraft traveling across the continent or across an ocean travel at typical heights up to thirty-nine thousand feet (39,000 ft). The in-cabin pressure typically corresponds to an altitude of five to six thousand feet (5,000-6,000 ft), equating to an FiO2 of seventeen and one-half percent (17.5%). For certain long-haul flights, an individual could be at that altitude for up to sixteen hours (16 hrs). A small study by Humphreys et al., considers the effect of high altitude commercial air travel on oxygen saturation (See Anesthesia, 2005, pp. 458-460), and shows that there is a significant reduction at cruising altitude of SpO2 in all passengers in the clinical study. The mean at ground level for the passengers was ninety-seven percent (97%) SpO2 while at cruising altitude the mean SpO2 dropped to ninety-three percent (93%) with several passengers experiencing drops in their SpO2 below ninety percent (90%).

Little attention has been focused on the dangers of lower oxygen partial pressures on aircraft and the impact on passengers. Most attention has been focused on deep vein thrombosis (“DVT”) and pulmonary emboli causing sudden death in people after traveling on aircraft. Most research on the potential cause of DVT has focused on the lack of mobility on aircraft and dehydration of passengers. As the study by Humphreys points out, however, the three predisposing factors for DVT are reduced blood flow, increased coagulability and damaged vessel walls. Limb oedema is believed to be greater in aircraft than for equivalent periods of times in cars or trains and worse with lower cabin pressures. Local hypoxia causes vasodilation and increased capillary permeability. Acute hypobaric hypoxia is known to induce a hypercoagulable state.

Another variable impacting the delivery of supplemental oxygen and maintaining a healthy SpO2 is mobility of the patient. Moderate exercise by COPD patients can improve the following: the body's use of oxygen, energy levels, anxiety, stress and depression, sleep, self-esteem, cardiovascular fitness, muscle strength, and dyspnea (shortness of breath). A GPS unit or a GPS unit in a patient's phone, along with the pulse oximeter 22 and a heart rate monitor, can provide significant valuable information to a healthcare provider and data collection for the POC 14 that may utilize the information to drive operation of the POC 14. The LOC monitor 4 may provide additional biometric data on the level of sleep and brain activity of the patient that can also be utilized by the POC 14 to drive operation based on the specific patient and learned habits or conditions of the patient.

Prior art devices discuss adjusting oxygen delivery from a cylinder of concentrated oxygen in response to a reduction of SpO2 shown on a pulse oximeter attached to a patient. Such algorithms are not practical for POCs. Moreover, to enhance treatment of a patient recovering post-operatively or suffering from a pulmonary disease such as pulmonary fibrosis, COPD or a cardiology disease that requires supplemental oxygen, data from the device, such as the POC 14, the LOC monitor 4, GPS, CPAP machine 18, pulse oximeter 22, health tracker device 12, medication device 24 and related healthcare devices, monitors and sensors, patient and environment are preferred to effectively and efficiently improve the health of the patient.

For example, having a device that monitors SpO2 attached to a patient, such as the pulse oximeter 22, that simply increases or decreases the flow of oxygen based on the SpO2 data may provide a short term benefit, but the recording of such information provides little to no information for the physician or respiratory therapist to help improve the health of the patient. Certain prior art devices adjust oxygen flow, but no data is provided to the caregiver, medical professional or an AI database to evaluate a more efficient treatment plan for the patient. Assuming, for example, a patient's SpO2 decreases from ninety-two percent (92%) to eighty-six percent (86%), the preferred embodiment of the system 2 records the event time, duration and place, preferably via GPS, but then also includes the additional information, such as heart rate, current FiO2 being delivered, altitude, movement of the patient, level of consciousness, breathing rate and additional available data. For the FiO2, the bolus size, oxygen purity and the input tidal volume are preferably measured, collected and analyzed. This information is all preferably downloaded to a patient database on the memory device 11. In addition, data on the disease or diseases suffered by the user would be provided in the database of the memory device 11 that is in communication with the master software application or central processor 42.

A caregiver, healthcare provider or other participant could review the data provided by the patient or the preferred system 2 and the collected data from the devices associated with the preferred system 2 over a length of time that is stored on the memory device 11. The information stored on the memory device 11 can be used by a healthcare provider or by the preferred system 2 itself to alter a patient's healthcare plan. If the collected and provided data reveals that at a certain time everyday a patient's SpO2 decreases significantly for two hours (2 hrs), while heart rate and location doesn't change, the healthcare provider may assume that the SpO2 drop is not due to physical activity. A review of the data collected from the LOC monitor 4 may reveal that the patient was sleeping. Further data from POC 14 may contain alarm codes, including, but not limited to, low battery, low oxygen, and no breath detected. A no breath detected alarm may suggest that the patient has fallen asleep and either the nasal cannula has fallen out or the patient has shallow inhalation that does not trigger the negative pressure sensor K. The health care provider or the preferred system 2 can then suggest an improved treatment plan of changing the oxygen flow during a nap from pulse dose to continuous flow.

Any device that perceives its environment and takes actions that maximize its chance of successfully achieving its goals may be considered a device utilizing AI. A more elaborate definition characterizes AI as a system's ability to correctly interpret external data, to learn from such data, and to use the lessons learned from the acquired data over time to achieve specific goals and tasks through flexible adaptation. In the case of the software applications 28, 30, 32, 34, 36, 38, 40 that may be employed in the CPU 10, a goal may be to maintain a specific SpO2 range while maximizing battery power conservation. For example, oxygen demand typically increases when a patient is exercising. The pulse oximeter 22 may send a signal to the CPU 10 or the processor N on the POC 14 that SpO2 is decreasing. In response, the CPU 10 or the processor N preferably communicates to the POC 14 to increase oxygen flow to the patient.

In the preferred embodiment, the processor N and software applications 28, 30, 32, 34, 36, 38, 40 evaluate the additional variable data collected before deciding to provide more oxygen to the patient. Moreover, the preferred processor N and software applications 28, 30, 32, 34, 36, 38, 40 evaluate the additional variable data in order to anticipate oxygen needs of the patient. For example, as variable data is collected regarding patient heart rate, altitude and movement via GPS located on POC 14, CPU 10, fitness tracker 12 or other hardware, the processor N or software applications 28, 30, 32, 34, 36, 38, 40 can calculate that there will likely be an increase in oxygen demand and send instructions to the POC 14 to begin increasing oxygen flow to the patient. Anticipating oxygen demand is preferably incorporated by the system 2 based on the machine learning or AI incorporated into the processor N and the software applications 28, 30, 32, 34, 36, 38, 40.

Initially, the software applications 28, 30, 32, 34, 36, 38, 40 and the processor N or other hardware preferably stores the variable data and provides additional or decreased oxygen flow based on information provided by the pulse oximeter 22. As more variable data is stored in the memory of the CPU 10, the cloud or other hardware associated with the system 2, a database for smart assessment of oxygen needs for the particular patient will develop from the system 2. Instead of rapidly responding to an SpO2 decrease, causing increased power consumption, evaluation of heart rate, mobility, location or other considered factors monitored by the preferred system 2 will allow the POC 14 to gradually increase or decrease oxygen flow before a spike in demand occurs and to preferably short circuit any extreme conditions that may be encountered by the patient, particularly with respect to SpO2 levels.

Additional anticipated health events that can be mitigated or averted through machine learning with the preferred system 2 include increasing RPM of the compressor D to increase oxygen purity in situations where a patient is traveling on an aircraft or traveling to a higher elevation where lower barometric pressure causes SpO2 to decrease. Depending on oxygen concentration levels, the software applications 28, 30, 32, 34, 36, 38, 40 or the processor N, may actuate an increase in the bolus size or flow (if continuous flow) of oxygen instead of increasing RPMs of the compressor D at a lower setting.

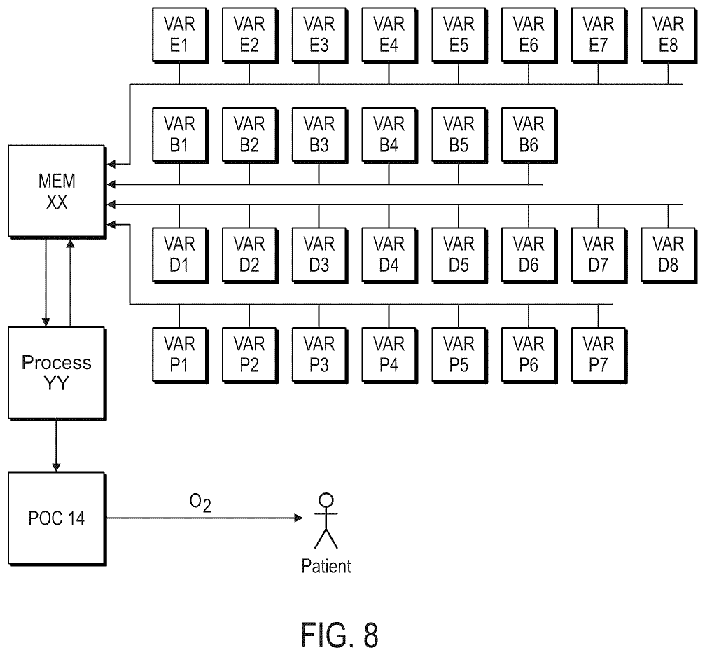

Referring to FIG. 8, a schematic diagram of components of portions of the preferred integrated system 2 and interaction between the components of the integrated system 2 of FIGS. 1, 6 and 7 with examples of stored and acquired data associated with various components of the preferred system 2 is depicted.

The POC 14 preferably senses, records or stores concentrator variables associated with the POC 14 and/or the patient during use. The concentrator variables are preferably sensed, recorded and/or stored and then sent to a memory of the system 2, such as in the CPU, central processor 42 or tablet 10. The concentrator variables may include, but are not limited to, environmental variables such as, but not limited to, time (E1), duration (E2), location (E3), temperature (E4), movement (E5), humidity (E6), pollution or pollen index (E7), altitude (E8) and other variables relevant to the POC 14 and/or the operation of the POC 14. Environmental variables can also be recorded by receivers on the POC 14 or provided by devices such as, for example, a mobile phone, smart watch 12, computer, Amazon Alexa or Google Hubs, a fitness tracker or other hardware that may be employed with the preferred system 2. Environmental variables can also be manually entered into a memory associated with the system 2 or automatically collected from known sources that may be in communication with the system 2, such as weather services, government data tracking services or other related services. Depending on the level of detail desired from an environmental variable, data may be collected once, continuously, at a set time or when a threshold is met or exceeded such as a temperature, altitude, humidity, barometric pressure limit, pollen index, ultraviolet (“UV”) index or other related variables that may be sensed and recorded.

Variables associated with the POC 14, which are typically acquired and collected from sensors associated with the system 2, may include, but are not limited to, oxygen purity (D1), oxygen flow (D2), oxygen bolus size (D3), battery power or power remaining (D4), power consumption (D5), breathing rate (D6), compressor RPM (D7), alarm codes (D8) and other variables that may be sensed and collected with respect to the POC 14 and the system 2.

Variables relating to patient biometric data, which are also typically acquired and collected from sensors associated with the system 2, may include, but are not limited to, data (B1) from the LOC monitor 4, heart rate (B2), blood pressure (B3), body temperature (B4), blood oxygen saturation or SpO2 (B5), tidal volume, other lung volume measurements (B6) and other related variables from the system 2 relating to patient biometric data.

Variables relating to patient personal data, which are typically provided by the patient, are included in the patient data, and updated as changes to the patient's personal data changes, may include, but are not limited to, age (P1), Sex (P2), Weight (P3), Disease (P4), O2 prescription (P5), SpO2 saturation target (P6), height (P7), race (P8) and related patient medical history.

On a very basic and initial level, a processor YY of the POC 14 may be configured to adjust oxygen flow to a patient in response SpO2 information received from the pulse oximeter 22 or other devices associated with the system 2 providing SpO2 information regarding the patient. The processor YY is preferably in communication with the CPU or tablet 10 and/or the central processor 42. As environmental, device, biometric and patient variable data is acquired and received into the memory 11 and the central processor 42, the data is preferably stored such that correlations and prediction models can be created based on the patient's history. As patterns and correlations are developed, the processor or CPU 10 can anticipate the oxygen needs of the specific patient associated with the POC 14. In addition, the processor YY is preferably able to distinguish between oxygen demand changes and device and patient errors that may occur during normal use of the preferred system 2. A low oxygen alarm may be caused by an altitude or barometric change, requiring the processor YY or memory device 11 to either increase the revolutions per minute of the compressor D or adjust the bolus size upward for delivery to the patient. In another preferred embodiment, the processor YY, central processor 42 or memory device 11 may decrease oxygen flow until the SpO2 collected from the pulse oximeter 22 is at the lower range for a patient in order to conserve battery power if the CPU 10 recognizes the patient is away from a home location where additional batteries 60 or AC power is located. In another preferred example, level of consciousness data from the LOC monitor 4 that is acquired from a consciousness sensor or sensors is provided and may suggest the patient is falling asleep or otherwise the patient's level of consciousness. The POC 14 may have capability to produce both pulse dose and continuous flow capabilities and could be configured to automatically change the method of delivery of the oxygen from pulse dose to continuous if prior data shows a correlation between sleep and no breath detected alarms.

The central processor 42 is configured to acquire data from the POC 14, the biometric sensor and the mobile device 10 and adjust the POC 14 based on comparisons of the biometric data point, the patient data and the flow data from the POC 14. The central processor 42 is preferably able to adjust the POC 14 based on predictions developed by artificial intelligence of the central processor 42 associated with the comparisons of the biometric data point, the patient data and the flow data, as is described herein.

The preferred embodiment of the system 2 will grow its database of acquired data with patient changes, providing significant trend and incident data for a caregiver that may be stored in the CPU 10, the memory device 11, the central processor 42 remotely or otherwise for comparison to other patients or to facilitate the machine learning and AI to improve patient outcomes. Currently, a healthcare provider typically relies on the memory of the patient, many who are elderly, to explain why their POC had to provide more oxygen on a particular day at a particular time. With such data also being capable of being transmitted remotely, a healthcare provider could set the processor YY, memory device 11 or CPU 10 to communicate with the master software application or central processor 42 such that medication is delivered each time an acute respiratory event occurs that requires bronchodilation. Such events are typically predicated by increased breathing rate and increased heart rate. The preferred system 2 may also check for air pollution or a pollen index variable E7 forecasted for a certain day that can be set to alert both the patient and healthcare provider that different oxygen or medication needs might be required and may provide an alarm or warning to the patient through the system 2.

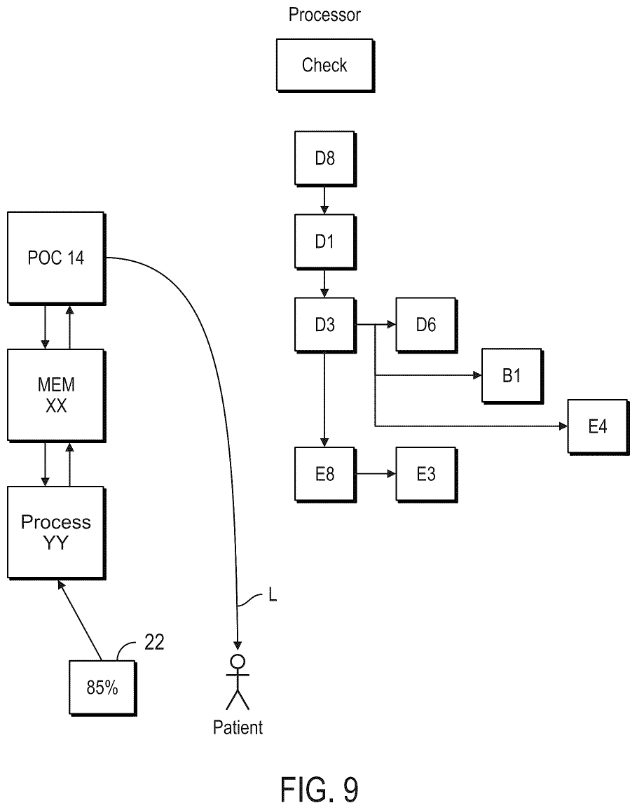

Referring to FIG. 9, a schematic of data flow and potential responses by the POC 14 to variable data involving the pulse oximeter 22 to improve a patient's health and recovery is depicted. A patient connected to the POC 14 via a nasal cannula L is also preferably, releasably attached to the portable pulse oximeter 22 with wireless communication, Bluetooth or a tethered communicating technology. The pulse oximeter 22 displays or collects data, for example, an SpO2 of eighty-five percent (85%). This data is transmitted to the processor YY, the memory device 11, the CPU 10 or other hardware of the system 2. As stated earlier, the processor YY can be a standalone processor on a tablet or phone associated with the CPU 10 that communicates with a software app on the processor of the POC 14. The processor YY may alternatively be comprised of a processor internal to the POC 14.

Before increasing oxygen flow to the patient to return their SpO2 to a preset or target level or a range established by the health provider, the processor YY, memory device 11 or CPU 10 reads the stored data in the memory device 11 or CPU 10 to compare variable data that has been stored on the memory device 11 or CPU 10. In the preferred embodiment of FIG. 9, in step 1 alarm variables D8 are reviewed for potential alarm codes such as low oxygen or no breath detected. If no relevant alarm codes are found, the preferred next step for the system 2 is to review a variable D1 for oxygen purity. If the oxygen purity has not dropped significantly enough to cause the recent drop in SpO2, the processor YY or CPU 10 preferably searches the memory device 11 for another variable D3, which is preferably bolus size. A cross check of the variables D6 (breathing rate), B1 (heart rate) and E4 (movement) may only show slow movement and slightly elevated heart rate in this preferred example. The next step for the preferred system 2 in response to the data reviewed is to check for variable E8, which is comprised of altitude. A lower barometric pressure corresponding to approximately five thousand four hundred feet (5,400 ft) has been recorded in this preferred example. A cross check with variable data E3, which is preferably comprised of location or GPS, shows the patient is in a suburb of Boulder, Colorado. The effective oxygen percentage in this preferred example is approximately seventeen and three tenths percent (17.3%). If the oxygen setting for the POC 14 can be raised, i.e., it is not at a maximum oxygen flow setting, the processor YY or CPU 10 preferably then sends a signal to the POC 14 to increase the flow setting upwards to compensate for the lower barometric pressure. Alternatively, if the POC 14 has the capability to adjust the compressor D revolutions per minute in order to process more air, then the processor YY or the CPU 10 may send a signal to the POC 14 to increase the revolutions per minute of the compressor D until a lower altitude is measured by the altitude sensor of the system 2. Alternatively, if in step 1 of this example variable D8 showed an alarm code of low or no oxygen or in step 2 and the variable D2 showed no breath detected, the processor YY or CPU 10 could send a signal to the POC 14 to increase the sound of the alarms to alert the patient that the cannula became kinked or disconnected or the sieve assembly G has failed.

A non-invasive breathing device 18 may also be utilized with the preferred embodiment of the system 2. The non-invasive breathing device 18 may be comprised of various devices and systems, such as, for example, continuous positive airway pressure (CPAP) and bi-level positive airway pressure (BiPAP) machines. CPAP and BiPAP machines are typically used as treatments for patients 8 suffering from sleep apnea. A CPAP machine increases air pressure in the patient's throat so that the airway does not collapse during an inhalation step of breathing. The CPAP/BiPAP 18 preferably includes a hose 20 that provides a conduit for pressurized air from the CPAP/BiPAP 18 to travel to the person or patient 8. Typically, the patient or person 8, would need a mask (not shown) that is connected at an external surface to the hose 20 wherein the mask is secured in a sealed manner via elastic straps to the face of the person 8 such that both the mouth and nasal openings of the person 8 are enclosed within the mask preventing the pressurized air from the CPAP/BiPAP 18 from leaking out between the face of person 8 and the mask.

Similar to the POC 14, the preferred CPAP/BiPAP 18 is able to transmit data to a software application 36 downloaded onto the tablet 10.

In situations where the person or patient 8 needs the use of supplemental oxygen from the POC 14 and pressurized air from the CPAP/BiPAP 18, the cannula 16 can be connected directly to the CPAP/BiPAP 18 or the hose 20 instead of to the person 8 such that the POC 14 and CPAP/BiPAP 18 operate in series. Concentrated oxygen may be delivered via the hose 20 to the person 8. The POC 14 and CPAP/BiPAP 18 may alternatively be connected to a common cannula 16 or hose 20 that feed pressurized air and concentrated oxygen directly to the patient 8, such that the POC 14 and CPAP/BiPAP 18 function substantially in parallel to each other.

A pulse oximeter 22 may also be utilized with the integrated system 2 and is shown removably attached to the finger of the person or patient 8 in FIG. 1. Generally, the finger pulse oximeter 22 functions by shining light through a patient's 8 finger. The pulse oximeter sensor 22 detects how much oxygen is in the patient's 8 blood based on the way the light passes through the finger. Pulse oximetry is the technology calculating the results to display a number on the oximeter's screen that tells a person the percent of oxygen in their blood. The finger pulse oximeter 22 typically also measures pulse rate. Examples of pulse oximeters 22 include ChoiceMed Fingertip by Concord, Vivosmart 4 by Garmin, the Zacurate premium fingertip pulse oximeter, and the MightySat by Masimo. The preferred pulse oximeters 22 may communicate via wired or wireless (Bluetooth) communication protocol with the tablet 10.

A study published in the Dec. 17, 2020, New England Journal of Medicine, Racial Bias in Pulse Oximetry Measurement, by Sjoding et al. renewed concerns regarding the accuracy of pulse oximeters with darker skinned patients. The accuracy of pulse oximeters with dark skinned patients is not a new problem. Racial bias in pulse oximetry was the subject of two studies in 2005 and 2007. In the 2020 study, black patients had nearly three times the frequency of occult hypoxemia when compared to the frequency of occult hypoxemia of white patients in substantially the same circumstances. Given the widespread use of pulse oximetry for medical decision making, these findings have significant implications, especially during the current coronavirus disease, 2019 (Covid-19) pandemic. The authors' results in the pulse oximetry study suggest that reliance on pulse oximetry to triage patients and adjust supplemental oxygen levels may place black patients at increased risk for hypoxemia.

As described herein, race P(8) is a variable that is preferably factored into the system 2 when designing or using the pulse oximeter 22. Utilizing an oximeter 22 that has not been adjusted or normalized to adjust SpO2 readings to reflect race or, more specifically, skin color, could lead to less favorable patient recovery.

Supplemental oxygen is administered in the vast majority of patients in the perioperative setting and in the intensive care unit to prevent the potentially deleterious effects of hypoxia. On the other hand, the administration of high concentrations of oxygen may induce hyperoxia that may also be associated with significant complications. Oxygen therapy should, therefore, be precisely titrated and accurately monitored. Although pulse oximetry has become an indispensable monitoring technology to detect hypoxemia, its value in assessing the oxygenation status beyond the range of maximal arterial oxygen saturation, which may be considered at over ninety-seven percent (SpO₂≥97%) is very limited. In the hyperoxic range, physical blood gas analysis is preferably performed, which is intermittent, invasive and sometimes delayed. The oxygen reserve index (ORI) is a new continuous, non-invasive variable that is provided by a new generation of pulse oximeters, potentially from Masimo, that use multi-wavelength pulse co-oximetry. The ORI is a dimensionless index that reflects oxygenation in the moderate hyperoxic range of partial pressure of oxygen of one hundred to two hundred millimeters of Mercury (PaO₂ 100-200 mmHg). The ORI may provide an early alarm when oxygenation deteriorates well before any changes in partial pressure of oxygen (SpO₂) occur, may reflect the response to oxygen administration (e.g., pre-oxygenation), and may facilitate oxygen titration and prevent unintended hyperoxia. The Masimo Root™ monitoring system is an example of a device that can measure ORI, pulse oximetry, brain function and other biometrics.

Hypoxemia is a below-normal level of oxygen in your blood, specifically in the arteries. Hypoxemia is a sign of a problem related to breathing or circulation, and may result in various symptoms, such as shortness of breath, skin color changes, confusion, cough, increases heart rate, sweating and other symptoms. Hypoxemia is determined by measuring the oxygen level in a blood sample taken from an artery (arterial blood gas). Hypoxemia can also be estimated by measuring the oxygen saturation of your blood using the pulse oximeter 22. Normal arterial oxygen is approximately seventy-five to one hundred millimeters of mercury (75-100 mm Hg). Values under sixty millimeters of Mercury (60 mm Hg) usually indicate the need for supplemental oxygen. Normal readings from the pulse oximeter 22 usually range from about ninety-five to one hundred percent (95-100%). Values under ninety percent (90%) are considered low.

In a preferred embodiment of the integrated system 2, the pulse oximeter 22 is connected via a tether or wirelessly, such as Bluetooth communication protocol, to the tablet 10 such that blood oxygen saturation level and pulse of the patient or person 8 can be transmitted to a software application 38 on the tablet 10 that stores in memory or the Cloud the blood oxygen level and pulse rate of the patient or person 8.

In a further preferred embodiment of the system 2, a Medication Device 24 is provided. The medication device 24 is shown as an intravenous (IV) bag connected via a catheter 26 to the patient or person 8. Other examples of medication devices 24 include, but are not limited to, syringes, infusion pumps, transdermal patches, nebulizers, inhalers, drug coated stents, nasal sprayers, and autoinjectors. Additional drug delivery devices include those described in International Patent Application Publication No. WO 2018/226532, titled, “Configurable Oxygen Concentrator and Related Method” and International Patent Application Publication No. 2017/165749, titled, “Positive Airway Pressure System with Integrated Oxygen,” each of which is incorporated herein by reference in their entirety. The medications being delivered via the medication device 24 can include, but are not limited to, bronchial dilators, antibiotics, anesthetics, and pain killers.

In the preferred embodiment, the medication device 24 may be comprised of a programmable infusion pump that can be operated or modified to operate remotely. Examples of such pumps include the SynchroMed II by Medtronic Corporation or Perfusor Space 2nd Generation Syringe Pump by B. Braun. In the preferred embodiment, the medication device 24 is connected via a tether or wirelessly, Bluetooth for example, to the tablet 10. A software application 40 on the tablet 10 stores in memory or in the Cloud, information or data received from the medication device 24. In a further preferred embodiment, a user of the tablet 10 through a software application 40 stored on the tablet 10 that received data from the medication device 24 can send instructions to operate or control the medication device 24. Such control could include the delivery or adjustment of the delivery of medication via medication device 24. The tablet 10 may also send signals and warnings to the patient 8 indicating that the software application 40, through artificial intelligence, deep learning, machine learning or neural networks, predicts potential medical issues for the patient 8, potential failures of the component mechanisms of the system 2 or requirements to contact a physician or caregiver. The software application 40 may utilize learning by artificial intelligence or deep learning based on review of the acquired data from the concentrated oxygen source 14, the LOC monitor 4, the health tracker device 12, the CPAP machine 18, the pulse oximeter 22, the medication device 24 and related healthcare devices, monitors and sensors, such as GPS sensors that track movement of the patient 8.

Personal information relating to the patient or person 8 is stored preferably in a memory device or central processor 11. The memory device 11 can be part of the tablet 10 or remotely stored on a universal serial bus (USB) drive, the Cloud or another computer system. Personal information on the memory device 11 could include age, weight, height, gender, medical history, employment history, deoxyribonucleic acid (DNA) information, and medications being taken by patient or person 8. Personal information of patient or person 8 can be entered manually on the memory device 11 contained in the tablet 10, transmitted from a portable memory device such as a thumb drive or downloaded from an external source such as a Cloud network. Depending on security settings established by the patient or person 8, the personal information on the memory device 11 could be accessed by the software applications 28, 30, 32, 34, 36, 38, 40 for implementation with the artificial intelligence, deep learning or neural networks for predicting healthcare needs of the patient 8.

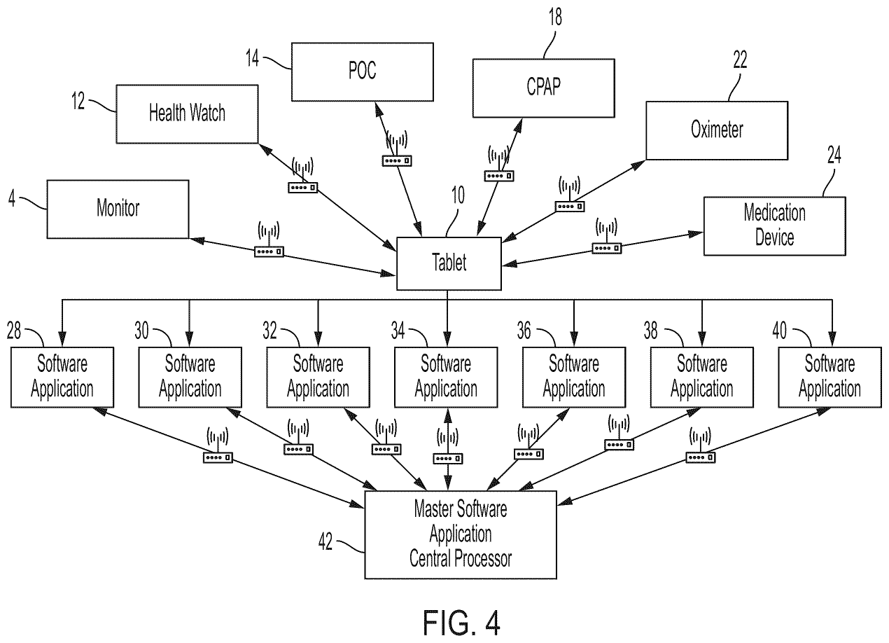

In a preferred embodiment, as shown in FIG. 4, a user of the tablet 10 can control one or more of the apparatus or devices connected to tablet 10 via software applications 28, 30, 32, 34, 36, 38, 40. In a further preferred embodiment, a separate controlling software application 42, herein known as the master software application 42, on the tablet 10 integrates or otherwise incorporates the data, information and ability to control from one or more software applications 28, 30, 32, 34, 36, 38, 40 on the tablet 10 such that a user of the tablet 10 can control all software applications through a single master software application 42. The master software application 42 would also have access to personal information stored on the memory device 11, either directly or through the software applications or tablet 10.

Where the user of the tablet 10 is a doctor or other caregiver, adjustments to the POC 14, the CPAP 18 or the medication device 24 can be made remotely based on data displayed in the master software application 42. For example, improving postoperative sleep has been shown to improve patient recovery and reduce patient readmissions. A caregiver reviewing the master software application 42 could review a sleep cycle chart or sleep pattern 46 generated from data transmitted from the LOC monitor 4 and remotely adjust the POC 14, the CPAP 18 or the medication device 24 in attempts to enhance or improve the sleep cycle of the person or patient 8, such as by increasing the purified oxygen flow from the POC 14 or increasing the pressure of the discharge air from the CPAP 18. In addition, the caregiver may alternatively adjust the ventilator 13 to incorporate or utilize concentrated oxygen from the POC 14 into the airflow of the ventilator 13 to enhance the patient's sleep cycle, which can be monitored by the LOC monitor 4 Improving the sleep cycle of the person or patient 8 could also be achieved by increasing the level of supplemental oxygen delivered by POC 14. It has been shown that increasing oxygen concentration levels helps patients 8 reach and lengthen their stage 3 and 4 sleep. In addition, a caregiver controlling the tablet 10 and the CPAP 18 may adjust the settings on CPAP 18 because of data received from the health watch 12, the pulse oximeter 22 or the monitor 4. In another example, data received from the monitor 4 could suggest the person 8 is experiencing excessive pain that is preventing meaningful sleep. The caregiver, using the tablet 10 could adjust pain medication delivered via the medication device 24, improving the likelihood of the person 8 improving their sleep quality. It is also envisioned that a caregiver could review the data received and displayed by the master software application 42 and make a variety of adjustments to one or more of the POC 14, the CPAP 18, the ventilator 13, or the medication device 24 to improve the sleep quality of person 8. The master software application or central processor 24 may also send alerts to a physician, the caregiver or the patient 8 to provide suggestions for treatment modification or follow-up based on analysis of the acquired data and the patient's 8 medical history using integral artificial intelligence, deep learning and cognitive computing in the central processor 24.

In another embodiment of the integrated system 2, the tablet 10 is a central processing unit (“CPU”) at a nursing or other caregiver work station capable of storing, operating, analyzing and displaying multiple master software applications 42 that can receive, process and control multiple software applications 28, 30, 32, 34, 36, 38, 40. A single caregiver could then monitor data, and remotely control multiple different POCs 14, CPAPs 18, ventilators 13 or medication devices 24 connected to multiple persons or patients 8 and also monitor each of the patient's 8 sleep cycle via the LOC monitor 4. In a further preferred embodiment of the integrated system 2, the CPU 10 is located hundreds or even thousands of miles from the person or patient 8 such as, for example, a base hospital in the USA and a small military hospital closer to a combat zone or on a ship. Another preferred example would be locating the CPU 10 in a hospital in a large metropolitan area with the patients 8 located in a distant rural area with limited caregiver resources.

It is further contemplated that a user of the master software application 42 would have access to the internet and external databases to assist with diagnosis or treatment of the patient or person 8.

Another preferred embodiment of the system 2 includes the master software application 42 having the ability to perform artificial intelligence and deep learning of the data, including patient medical history or records, and information received from multiple software applications 28, 30, 32, 34, 36, 38, 40 or directly transferred from the monitor 4, the watch 12, the POC 14, the CPAP 18, the oximeter 22, the ventilator 13, the medication device 24 or other similar device that is able to acquire data related to the patient 8, such that the master software application 42 automatically adjusts and controls the POC 14, the CPAP 18, the ventilator 13, the medication device 24 or other related treatment mechanism to improve the sleep and postoperative recovery of the patient or person 8. The preferred system 2 also envisions that in situations where it would be illegal, unethical, unpractical or otherwise impossible to permit the master software application 42 to remotely adjust the POC 14, the CPAP 18, the ventilator 13, the medication device 24 or other treatment mechanism, the artificial intelligence or deep learning results performed by the master software application 42 could be generated as a suggested or recommended treatment protocol for a caregiver with access to the tablet 10, the POC 14, the CPAP 18, the ventilator 13, the medication device 24 or other treatment mechanism. The central processor 42 may also send notifications to the patient 8, the caregiver or the healthcare professional regarding suggestions for treatment, warnings related to the POC 14, the CPAP 18, the ventilator 13, the medication device 24 or other treatment mechanism related to required maintenance or scheduling of treatments with a physician based on analysis of the acquired data by the artificial intelligence, deep learning, machine learning and neural networks of the central processor 42.

The processing of deep learning and artificial intelligence calculations under the master software application 42 could also include data not only from person 8, but also a database created from storing information from multiple previous patients 8 who have undergone similar or the same procedures as the subject patient 8 in an external memory storage apparatus such as the Cloud. As the master software application 42 performs more adjustments to the POC 14, the CPAP 18, the ventilator 13 or the medication device 24 on various patients 8, more data is generated, thereby providing a deeper learning treatment database for further automated remote adjustments of the POC 14, the CPAP 18, the ventilator 13, the medication device 24 or other related equipment for new person 8.

Information that would be collected and stored in a treatment database would include, but not be limited to, name, age, height, weight, gender, race, location, diseases, habits such as smoking or drug use, cholesterol level, injuries, surgeries, DNA, employment history, calories burned, diet, pulse, oxygen saturation, blood pressure, oxygen concentration, oxygen flow rate, pulse dose or continuous flow, CPAP or BiPAP use, flow level, pressure settings, medications, drug delivery type, breathing patterns, and environmental conditions.

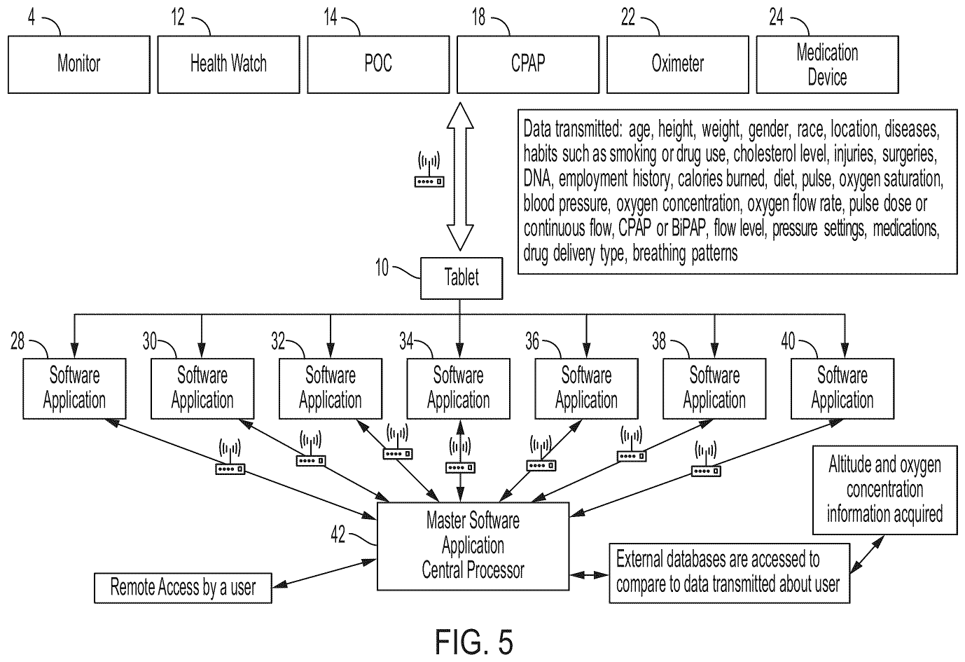

An example of a preferred embodiment of the invention is shown in FIG. 5. Data is acquired and transmitted by the monitor 4, the watch 12, the POC 14, the CPAP 18, the oximeter 22, the medication device 24 or other related health mechanism, such as the ventilator 13, that is preferably connected to the patient or person 8 postoperatively, to the tablet 10 and to the central processor 42. The tablet 10 has the software applications 28, 30, 32, 34, 36, 38, 40 loaded on it which are then connected to or are in communication with the central processor or master software application 42. The tablet 10 has wireless local area networking, Wi-Fi or cellular capability and preferably has access to the Internet. Databases are available or in communication with the central processor 42 that provide additional information regarding the environmental conditions of the location of the patient or person 8. Additional databases are available or in communication with the central processor 42 that contain information cross-referenced with other individuals and name, age, height, weight, gender, race, location, diseases, habits such as smoking or drug use, cholesterol level, injuries, surgeries, DNA, employment history, calories burned, diet, pulse, oxygen saturation, blood pressure, oxygen concentration, oxygen flow rate, pulse dose or continuous flow, CPAP or BiPAP, flow level, pressure settings, medications, drug delivery type, and breathing patterns.

The master software application or central processor 42 compares the data transmitted with information from known base data and utilizes its internal processing, artificial intelligence, machine learning, neural networks or deep learning that, for example, indicates the patient or person 8 has a blood oxygen saturation level below acceptable levels. A review of performance data of the CPAP 18, the ventilator 13 and the POC 14 may reveal, for example, an acceptable oxygen concentration level of ninety percent (90%). The central processor or master software application 42 may access environmental databases and other relevant databases for comparative analysis. The environmental database may reveal that the patient or person 8 is located at a position ten thousand feet (10,000′) above sea level. At this elevation, the effective oxygen level is fourteen and three tenths percent (14.3%) or nearly thirty percent (30%) less than at sea level. The central processor or master software application 42 preferably calculates the difference in additional supplemental oxygen and sends a command to the POC 14 or the ventilator 13 to increase the flow of oxygen accordingly and may prompt more frequent monitoring of the patient's 8 breathing while they are located in the greater elevation.

Another example of a preferred embodiment of the system 2 improves the outcome of patients recovering from an acute COPD exacerbation or heart surgery. It was shown in a study, Effect of Home Noninvasive Ventilation with Oxygen Therapy vs. Oxygen Therapy Alone on Hospital Readmission or Death After an Acute COPD Exacerbation, Murphy, Patrick et al, JAMA 2017:317(21):2177-2186, that patients with persistent hypercapnia recovering from an acute COPD exacerbation that utilize noninvasive ventilation therapy, such as a CPAP machine 18, with oxygen therapy, such as the POC machine 14, have a lower risk of readmission or death within one year after the exacerbation event.

It has further been shown in another study, The Severity of Sleep Disordered Breathing Induces Different Decrease in the Oxygen Saturation During Rapid Eye Movement and Non-Rapid Eye Movement Sleep, Eunkyung, Choi et al, Psychiatry Investigation 2016; 13(6):652-658, that in the case of simple snoring, the average oxygen saturation during REM sleep was statistically, significantly higher than in NREM sleep. Patients with mild and moderate obstructive sleep apnea syndrome (OSAS) showed no significant difference in oxygen saturation during REM and NREM sleep. In the case of patients with severe OSAS, the average oxygen saturation was lower during REM than NREM sleep. Previous studies reported that apnea or hypopnea can be further exacerbated during REM sleep, as compared to NREM sleep and that average oxygen saturation was lower during REM sleep, as compared to NREM sleep.

With artificial intelligence, deep learning or neural networks incorporated into the central processor 24, as is described herein, the above studies, inputs, data, results and other studies and associated inputs, results, data and conclusions, for example, involving sleep patterns, postoperative readmissions and death causes, causes of low oxygen saturation levels, COPD impact on breathing patterns and oxygen intake, sleep apnea levels and impact on breathing rate and oxygen intake, to name a few, can be inputted into the central processor 42. The integrated system 2, by incorporating the ability of the central processor or the master software application 42 to access the database 50, a typically non-linear learning procedure can be created wherein the central processor or master software application 42 compares and weighs the results of the data from the monitor 4, the POC 14, the CPAP 18, the ventilator 13, the oximeter 22 and the watch 12 with stored information. For a particular patient or person 8, a sleep pattern or chart 46, such as shown in FIG. 3, can be accessed from a time prior to a COPD exacerbation or heart surgery event with the monitor 4 that is stored in memory in the software application 28 and/or the central processor 42. In the event a specific patient or person 8 utilizes a CPAP 18 or a POC 14, data during the same sleep event can be stored in memory in the software application 36, 34, respectively.

Postoperatively, the patient or person 8 is then monitored under the integrated system 2. In the event hypercapnia occurs or the specific patient or person 8 shows low blood oxygen saturation levels, the central processor or master software application 42 can compare prior sleep pattern data with current acquired data and also the information of the patient's 8 sleep data from preoperative sleep to generate a recommended treatment. In addition, with prior or preoperative sleep pattern data, the central processor or master software application 42 can monitor a person 8 who has severe OSAS for dangerously low oxygen saturation levels during REM sleep if the prior sleep pattern shows such a pattern. Data transmitted from the LOC monitor 4 would alert the central processor or master software application 42 that the patient or person 8 was entering a REM period and the central processor or master software application 42 could begin to increase oxygen flow through the POC 14 or the CPAP 18 or a combination of both.

In another preferred example, the LOC monitor 4 may be incorporated into a mask (not shown) or associated with a mask of the POC machine 14, the ventilator 13 or the CPAP machine 18. A patient 8 who has a pre-existing condition that requires the POC machine 14 or the CPAP machine 18 is able to transmit acquired sleep information from the LOC monitor 4 to the central processor 42 prior to a procedure or preoperative such that the central processor 42 has a stored history of sleep data for the patient 8. The central processor 42 also preferably has access to baseline data regarding typical sleep data for a similar patient of similar age and having a similar medical history. The patient 8 may then experience a treatment, such as a surgical procedure. For example, the patient 8 may undergo a hip replacement. The central processor 42 is, thereafter, able to continue to monitor the sleep patterns of the patient following the surgery through the LOC monitor 4 associated with the POC machine 14, the ventilator 13 or the CPAP machine 18. The central processor 42 preferably acquires data related to the patient 8 postoperatively and compares the postoperative data, such as the sleep data, to the preoperative data. The central processor 42 is preferably able to adjust the POC machine 14, the ventilator 13 and/or the CPAP machine 18 to facilitate or aid the patient's 8 sleep patterns or transmits warnings or updates to the patient 8 and/or to the physician related to the patient's postoperative sleep patterns. The central processor 42 is also preferably able to push notifications to the patient 8 through the tablet 10 regarding therapies, such as movement or walking, medications, medical appointments or other information for the patient 8. The central processor 24 is also preferably able to compare the patient's postoperative sleep patterns to typical sleep patterns for patient's that have undergone the same procedure and have similar medical histories to track the patient's recovery in relation to a comparable patient 8.

As more and more patients use the preferred integrated health monitoring system 2, a growing, more detailed database of environmental, biometric, device and patient variables will provide new modeling opportunities for AI to predict healthcare events. Biometric data such as DNA can reveal certain populations of people with the same genome suffering similar events such as acute respiratory distress syndrome (“ARDS”) under certain conditions such when the pollen index is high, humidity is high, and temperature is high. As the patient population grows, the intelligence of the preferred integrated health monitoring system 2 also grows.

The central processor 42, which may be incorporated into the tablet, smartphone or computer 10, collects data from the POC 14, such as in APP or software designed to run on the tablet, smartphone, computer or other mobile device 10, so that the patient's 8 oxygen therapy data is available to the patient 8. The APP may track the patient's breath rate, oxygen usage (hours used), pulse volume, battery life and alarm history from sensors associated with the POC 14 and the patient 8 and the data is transmitted to the central processor 42. The APP may further provide maintenance reminders to the patient 8 through the mobile device 10, smart watch 12 or other device, such as reminders to replace the cannulas 16, the molecular sieve assembly G, a filter at the inlet B, other filters or other maintenance reminders, provide access to the user manual and/or provide the ability to input spirometer and pulse oximeter data by the patient 8, which is transmitted to the central processor 42 for review and analysis. The APP may also remind or warn the patient 8 regarding filter notifications, alarms, empty battery 60, charging status, low power for the battery 60, audio alarm status, breath detection of the patient 8, oxygen levels in the hose 20 or oxygen cannula 16 or other alarms. The mobile device 10, smart watch 12, POC 14, ventilator 13, LOC monitor 4, CPAP 18, pulse oximeter 22, consciousness sensor 6, medication device 24 and memory device 11 may be connected to each other and/or the central processor 42 via a wireless protocol, such as Bluetooth.

The central processor 42 may also collect data from third party devices via wireless communication protocols including pulse, heart rate, breathing rate, patient steps, location, altitude, global position, pollen index, air quality, sleep duration, sleep cycle, i.e. rapid eye movement (“REM”) sleep duration, blood oxygen saturation and other data collected about the patient 8 or the patient's environment. The system and central processor 42 preferably collects data from the POC 14 related to run time, intake filter time (to determine recommended replacement time), cartridge purity of the molecular sieve assembly G, breathing rate, power level of the batteries 60, remaining charge or life of the battery 60, error codes, alarms and other system data. The APP also preferably permits display of advertisements from the display 10a, which may be third party advertising that is paid advertising.

The data collected by the central processor 42 may be compiled and displayed on the display 10a, which may be on the mobile device 10 or other system for the patient 8, healthcare provider or other individual to monitor the patient 8 and the system. The data may be compiled into chart, tables, graphs or other visual representations to show trends or to assist with diagnosis of the patient 8. The charts, tables or graphs may show sleep duration versus blood oxygen saturation, blood oxygen saturation versus POC 14 run time or other comparisons for patient or system analysis.

The central processor 42 is preferably configured to compile the collected data from the sensors and as input by the patient 8 or other source and provide feedback to the patient 8. As a non-limiting example, the central processor 42 may receive blood oxygenation data from the pulse oximeter 22, which indicates a gradual decrease in blood oxygen saturation. The central processor 42 may send a signal to the mobile device 10 or smart watch 12 to display a warning/alarm or transmit an audible warning that the patient's blood oxygen saturation is dropping and the patient needs to increase their flow of oxygen. The patient or healthcare professional may set the preferred oxygen saturation level by increasing the output of the POC 14, the ventilator 13, the CPAP machine 18 or other device to increase the oxygen or air provided to the patient 8. For example, a typical COPD patient may have a preset range of eighty-nine to ninety-two percent (89-92%) desired blood oxygenation level. In another example, a heart patient may have a preset range of ninety-five present (95%) or higher.

The central processor 42 may also provide predetermined reports to the patient's 8 physician or other healthcare provider at predetermined timeframes.

The central processor 42, which may control, be in communication with or be comprised of the APP, may be designed and configured to automatically change the amount of oxygen supplied to the patient 8 based on the collected blood oxygen data and current purity of the oxygen being delivered. The central processor 42 may include predetermined protocols and ranges that establish the accuracy of the pulse oximeter 22 and any adjustment needed for the patient's skin color. The central processor 42 may further use machine learning/AI to begin adjusting the flow of oxygen based on the current purity level, increasing or decreasing heart rate, blood oxygen level, location of the patient 8 or another variable. For example, as a patient begins climbing in altitude, a higher level of oxygen will start flowing to the patient 8 based on collected location data in anticipation of the lower atmospheric pressure effectively causing the patient 8 to breath harder to acquire the same level of oxygen as at sea level. The same can be achieved during a sleep cycle resulting in an increase or decrease of oxygen level to the patient 8. For example, as patient 8 moves through the sleep cycle oxygen can be increased and decreased in response to previous sleep pattern data that was collected by the system and transmitted to the central processor 42. As an additional non-limiting example, the central processor 42 may increase or decrease the oxygen volume or purity provided to the patient 8 from the POC 14 based on pollen level data collected by the central processor 42 and historical patient data related to how the oxygenation level of the patient was impacted by similar high pollen levels. As a further non-limiting data, the central processor 42 may increase or decrease oxygen volume or purity provided to the patient based on the exercise level or ambulation of the patient 8 during a predetermined period of time.

The central processor 42 may be comprised of a dedicated central processor 42 that receives, compiles and analyzes data for an individual patient system or may receive, analyze and compile data from multiple systems and patients 8 to provide modifications to oxygen levels based on multiple experiences with patients 8. The central processor 42 may utilize the collected data from multiple patients 8 to identify trends for treatment purposes. The collect data from each patient 8, preferably is associated with information related to each of the patients 8, including age, weight, gender, location, medications used and other related patient information. The central processor 42 may, for example, analyze and compile collected data from various patients 8 to determine whether certain medications have a bigger impact (or no impact) on sleep, oxygen usage, mobility, blood oxygenation or other patient information.

The central processor 42 may also analyze how purity and volume of oxygen improve treatment for the patient 8 over time. As a non-limiting example, a patient 8 may be breathing at twenty breaths per minute (20 BPM). Their breathing cycle (inhale through exhale) is three seconds (3 sec). In addition, the patient 8 is receiving the equivalent of three liters (3 L) of pulse dose oxygen or six hundred milliliters (600 ml) per minute. This oxygen delivery equates to approximately a thirty milliliter (30 ml) bolus of oxygen every three seconds (3 sec). The central processor 42 may include a predetermined standard that thirty milliliters (30 ml) should be delivered in the first six tenths of a second (0.60 second) of the patient's breathing cycle. The central processor 42 may utilize machine learning and/or AI to determine whether the patient 8 respond better to a thirty milliliter (30 ml) of ninety-five present (95%) oxygen bolus, a forty-four milliliter (44 ml) bolus of sixty-five percent (65%) pure oxygen or another combination of volume and purity of oxygen. The central processor 42 may determine that the larger volume of lower concentrated oxygen “washes” carbon dioxide (CO₂) built up in the lungs/respiratory space based on collected sensor data and/or data collected from other patients in related systems. A treatment may also be developed that may be more applicable for certain lung disorders based on historical and patient data collected from the sensors.

Referring to FIGS. 1-11, the POC 14 provides supplemental oxygen to the patient 8 as part of the integrated system for monitoring conditions of the patient 8. The POC 14 is preferably a transportable, software-monitored device designed to be used by patients as a portable oxygen delivery system that delivers high concentrations of oxygen on a supplemental basis. The POC 14 is preferably small, portable and capable of continuous use in home, institutional, travel and mobile environments. The POC 14 preferably operates and is controlled for operation by the central processor 42 in a pulse dose mode and a no breath detected mode. In the pulse dose mode, the patient's inhalation and exhalation patterns are sensed and the central processor 42 directs the POC 14 to release a pulse of air or a bolus of air through the oxygen cannula 18 prior to the patient's inhalation. The central processor 42 preferably initially detects a patient's breath based on acquired pressure data in the oxygen cannula 18, a mouthpiece or an outlet of the POC 14 and provides a message or indication to the patient 8 that breath is detected. In the no breath detected mode, the central processor 42 determines that no breath is detected based on a lack of changing pressure in the oxygen cannula 18, the mouthpiece or an outlet of the POC 14 for a period of time, such as forty-five seconds (45 sec) and provides a message or an alarm to the patient 8 that no breath is detected. After an additional predetermined period of time, such as forty-five seconds (45 sec), the central processor 42 preferably sends an additional message or alarm that no breath is detected and automatically operates the POC 14 in a no breath mode wherein pulses or air or bolus are delivered every fifteen seconds (15 sec) at a rate of an anticipated fifteen breaths per minute (15 BPM). The central processor 42 is not limited to operating the POC 14 in the pulse dose mode and the no breath detected mode or in the manners described above and may operate in numerous other manners and protocols. For example, in the no breath mode, the central processor 42 may direct the POC 14 to deliver a continuous flow of oxygen to the patient 8 without pulses or a bolus.

The central processor 42 may transmit alarms to the patient 8 through the mobile device 10, the smart watch 12 or other device available to the patient 8 related to status or warnings related to the system 2. The alarm may be comprised of an icon on the display 10a or another display such as a display on the POC 14 and/or an audible alarm tone to draw the patient's attention. The alarms may indicate a malfunction of one of the components of the system 2, such as the POC 14, the level of consciousness monitor 4, the consciousness sensor 6, the memory device 11, the ventilator 13, the CPAP machine 18, the pulse oximeter 22, the battery 60 or other components of the system 2. The central processor 42 may utilize mathematical algorithms and time delays to reduce the potential for false alarms while providing alarms to the patient 8 when malfunctions of the system 2 occur. The alarms may include system shutdown, low battery, battery is disconnected and direct power is recommended, replace the battery 60, overheated system, low temperature system, low oxygen levels, replace the molecular sieve assembly G, battery 60 is not charging, lack of breath detection for a predetermined period of time, such as forty-five seconds (45 sec), sensor failure, oxygen delivery error and related alarms related to the system 2.

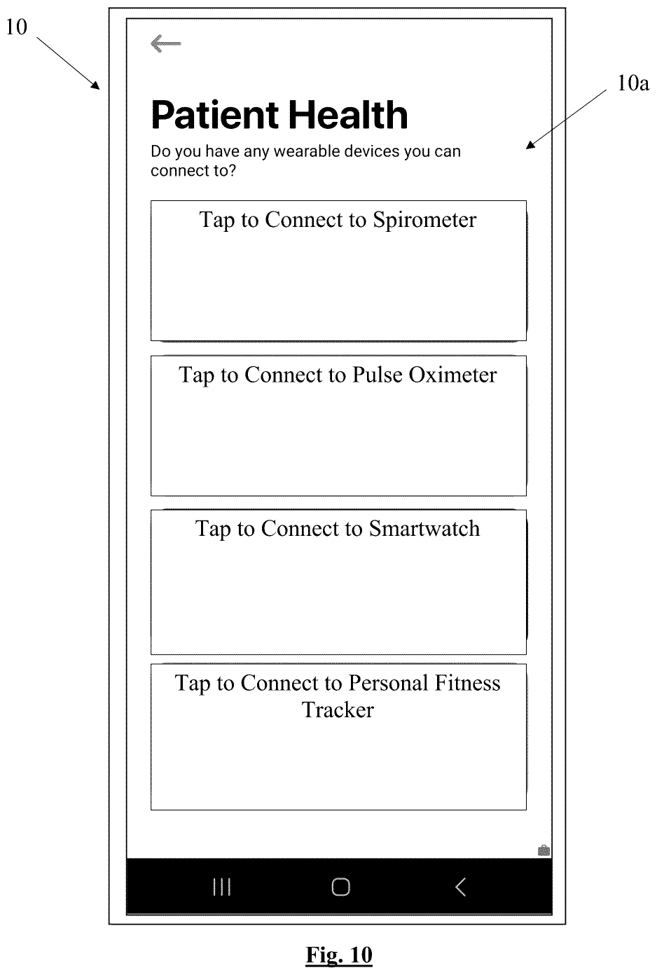

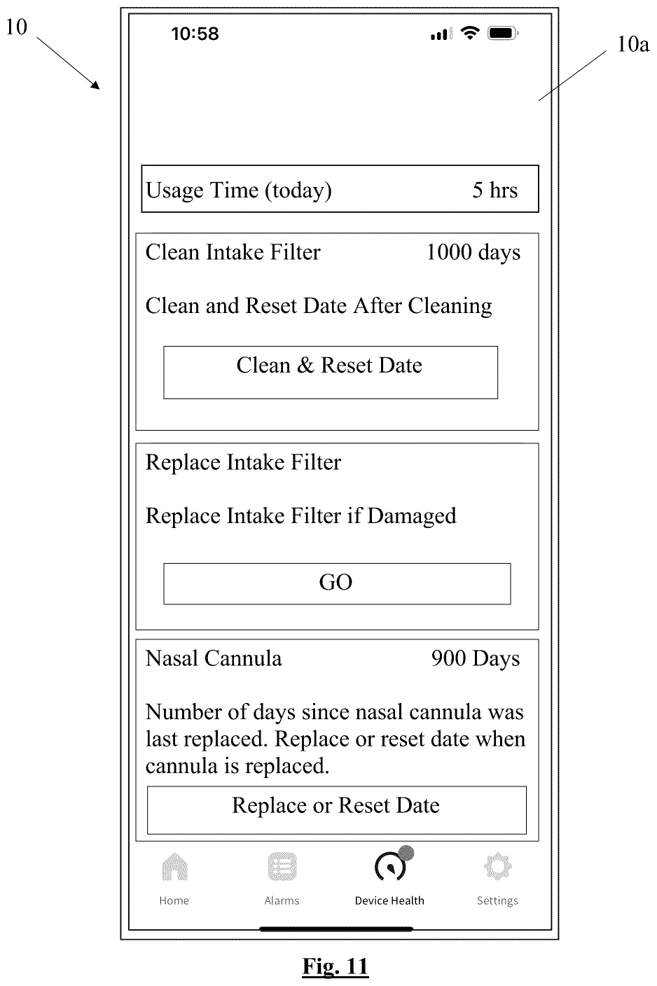

Referring to FIG. 10, when the central processor 42 is initially connecting to the system 2 and its components, a prompt may be provided to the patient 8 on the display 10a asking if the patient desires to connect the central processor 42 to components and/or sensors of the system 2. The patient 8 may request connection to a spirometer, which is used to measure the amount of air the patient 8 is able to breath in and out and tracks the time it takes to exhale completely after taking a deep breath, the pulse oximeter 22, the smartwatch 12, a personal fitness tracker and other components and sensors, such as the LOC monitor 4, the consciousness sensor 6, the memory device 11, the ventilator 13, a pressure sensor in the oxygen cannula 16, an oxygen purity sensor in the oxygen cannula 16, the CPAP machine 18, the mediation device 24, the POC 14 and related components and sensors. The central processor 42 may request or propose these connections only upon initial operation of the system 2, at each startup of the system 2 or based on a request by the patient 8 to search for components or sensors for connection. The central processor 42 preferably utilizes these connections to collect data to monitor the system 2 and its related components and to drive operation of the system based on control by the central processor 42.

Referring to FIG. 11, the system 2 may specifically collect and display data to the patient 8 on the display 10a or on an alternative display related to the POC 14 or any of the other components of the system 2. Specifically related to the POC 14 and as a non-limiting example, the central processor 42 may display usage time of the POC 14, the amount of time that the intake filter at the inlet B has been used with the POC 14 during operation, and the amount of time that the nasal cannula associated with the oxygen cannula 16 has been utilized while the POC 14 is operating. The central processor 42 may provide alerts or messages to the patient 8 on the display 10a regarding when to replace or clean the intake filter, when to remove and replace the molecular sieve assembly G, when to remove and replace the nasal cannula, when to remove and replace or recharge the battery 60 or other components based on predetermined run times of the components. The central processor 42 may also prompt the patient 8 to provide data to the central processor 42 through the display 10a regarding when the intake filter is cleaned, removed and/or replaced to restart the operation time and provide similar data input opportunities related to the nasal cannula, the battery 60, the molecular sieve assembly G and related components and features.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present disclosure.

I/We claim:

1. An integrated system for monitoring conditions of a patient, the integrated system comprising:
a central processor, the central processor including a sleep database including baseline sleep information for a generic patient having a similar medical history to the patient;
an oxygen concentrator configured to provide a flow of concentrated oxygen to the patient, the oxygen concentrator in communication with the central processor;
a level of consciousness monitor configured to collect data regarding the patient's state of wakefulness, the level of consciousness monitor in communication with the central processor; and
a mobile device configured for transport by the patient, the mobile device in communication with the central processor, the central processor collecting data from the oxygen concentrator, the level of consciousness monitor and the mobile communication device and adjusting the oxygen concentrator based on comparisons of the baseline sleep information and the collected data regarding the patient's state of wakefulness, the central processor adjusting oxygen purity and oxygen volume provided by the oxygen concentrator based on data collected from the integrated system.

2. The system of claim 1, wherein the oxygen concentrator is comprised of at least one of a portable oxygen concentrator and a continuous positive airway pressure machine.

3. The system of claim 1, wherein the mobile communication device is comprised of a tablet.

4. The system of claim 1, wherein the mobile communication device is comprised of a mobile phone.

5. The system of claim 4, wherein the mobile phone is configured to transmit movement data to the central processor based on global positioning system protocol.

6. The system of claim 1, wherein the central processor is in communication with a ventilator.

7. The system of claim 1, further comprising:
a medication device in communication with the central processor, the medication device configured to administer medication to the patient.

8. The system of claim 1, further comprising:
an oximeter in communication with the central processor.

9. The system of claim 1, wherein the central processor is configured to transmit an alert to the mobile device based on comparison of the collected data from the level of consciousness monitor and the baseline sleep information.

10. The system of claim 1, wherein the central processor is configured to predict potential medical issues of the patient by applying artificial intelligence to review acquired data from the oxygen concentrator and the level of consciousness monitor.

11. An integrated system for monitoring conditions of a patient, the integrated system comprising:
a central processor;
an oxygen concentrator configured to provide a flow of concentrated oxygen to the patient, the oxygen concentrator in communication with the central processor, the oxygen concentrator configured to sense volumetric flow data based on the flow of concentrated oxygen during use and oxygen purity data delivered from the oxygen concentrator, the oxygen concentrator transmitting the volumetric flow data and the oxygen purity data to the central processor;
a biometric sensor configured to sense a biometric data point of the patient during use; and
a mobile device configured for transport by the patient, the mobile device in communication with the central processor, the mobile device configured to sense patient data during use and receive the volumetric flow data and the oxygen purity data, the central processor configured to acquire data from the oxygen concentrator, the biometric sensor and the mobile device and adjust the oxygen concentrator based on comparisons of the biometric data point, the patient data, the oxygen purity data and the volumetric flow data.

12. The system of claim 11, wherein the biometric sensor is comprised of a pulse oximeter.

13. The system of claim 11, wherein biometric sensor is comprised of a negative pressure sensor.

14. The system of claim 11, wherein the biometric sensor is comprised of a consciousness sensor associated with a level of consciousness monitor, the biometric data point including electrical activity in the patient's brain.

15. The system of claim 11, wherein the patient data includes pulse/heart rate, body temperature, location, calories burned, distance travelled, time, steps, race, age, sex or mobility.

16. The system of claim 11, wherein the oxygen concentrator is configured to sense and record concentrator variables associated with the oxygen concentrator, the concentrator variables including time, duration, location, temperature, movement, humidity, pollution index, pollen index or altitude.

17. The system of claim 11, wherein the oxygen concentrator includes a battery, the patient data including oxygen concentrator data associated with the oxygen concentrator, the oxygen concentrator data including the oxygen purity data, oxygen bolus size, battery power, remaining battery power, battery power consumption, patient breathing rate, compressor revolutions per minute or alarm codes.

18. The system of claim 11, wherein the patient data includes a level of consciousness of the patient, patient heart rate, patient blood pressure, patient body temperature, patient blood oxygen saturation, patient tidal volume, and patient lung volume.

19. The system of claim 11, wherein the patient data includes age, sex, weight, patient disease indication, patient oxygen prescription, SpO2 saturation target, height, or race.

20. The system of claim 11, wherein the central processor adjusts the oxygen concentrator based on predictions developed by artificial intelligence of the central processor associated with the comparisons of the biometric data point, the patient data and the flow data.
